(19) United States
(12) Patent Application Publication
(10) Pub. No.: US 2011/0201118 A1
(43) Pub. Date: Aug. 18, 2011

(54) NUCLEASE ACTIVITY OF TAL EFFECTOR AND FOKI FUSION PROTEIN

(75) Inventors: BING YANG, Ames, IA (US); TING LI, Ames, IA (US); SHENG HUANG, Ames, IA (US)

(73) Assignee: IOWA STATE UNIVERSITY RESEARCH FOUNDATION, INC., Ames, IA (US)

(21) Appl. No.: 13/025,305

(22) Filed: Feb. 11, 2011

Related U.S. Application Data
(60) Provisional application No. 61/404,575, filed on Oct. 5, 2010.
(60) Provisional application No. 61/397,583, filed on Jun. 14, 2010.

(51) Int. Cl. C12N 15/01; C07K 14/00; C07H 21/00; C12N 5/10; C12N 15/63

(52) U.S. Cl. 435/441; 530/350; 536/23.4; 435/325; 435/320.1

(57) ABSTRACT

The present invention provides compositions and methods for targeted cleavage of cellular chromatin in a region of interest and/or homologous recombination at a predetermined site in cells. Compositions include fusion polypeptides comprising a TAL effector binding domain and a cleavage domain. The cleavage domain can be from any endonuclease. In certain embodiments, the endonuclease is a Type IIS restriction endonuclease. In further embodiments, the Type IIS restriction endonuclease is FokI.

### CROSS-REFERENCE TO RELATED APPLICATIONS

This application claims priority under 35 U.S.C. §119 to provisional application Ser. Nos. 61/397,583 filed Jun. 14, 2010 and 61/404,575 filed Oct. 5, 2010, herein incorporated by reference in their entirety.

### STATEMENT AS TO FEDERALLY SPONSORED RESEARCH

This invention was made with government support under Grant No. 0820831 awarded by the US National Science Foundation. The government has certain rights in the invention.

### TECHNICAL FIELD

This invention relates to methods for homologous recombination and gene targeting, and particularly to methods that include the use of transcription activator-like (TAL) effector sequences.

### BACKGROUND OF THE INVENTION

DNA double-strand breaking (DSB) enhances homologous recombination in living cells and has been exploited for targeted genome editing through use of engineered endonucleases, notably zinc finger nucleases (ZFN), a type of hybrid enzyme consisting of DNA binding domains of zinc finger proteins and the FokI nuclease domain (FN). Similarly, nucleases can also be made by using other proteins/domains if they are capable of specific DNA recognition.

The most significant application of endonucleases that are modified or custom-engineered to recognize longer DNA sequences is target genome editing in the post-genome era. The key component of the engineered nucleases is the DNA recognition domain that is capable of directing the nuclease to the target site of genome for a genomic DNA double strand break. The cellular DSB repair due to nonhomologous end-joining (NHEJ) results in mutagenic deletions/insertions of a target gene. Alternately, the DSB can stimulate homologous recombination between the endogenous target locus and an exogenously introduced homologous DNA fragment with desired genetic information, a process called gene targeting. The most promising method involving gene or genome editing is the custom-designed ZFN technology. The ZFN technology primarily involves the use of hybrid proteins derived from the DNA binding domains of zinc finger (ZF) proteins and the nonspecific cleavage domain of the endonuclease FokI. The ZFs can be assembled as modules that are custom-designed to recognize selected DNA sequences following binding at the preselected site, a DSB is produced by the action of cleavage domain of FokI.

The FokI endonuclease was first isolated from the bacterium Flavobacterium okeanokoites. This type IIS nuclease consists of two separate domains, the N-terminal DNA binding domain and C-terminal DNA cleavage domain. The DNA binding domain functions for recognition of a non-palindromic sequence 5′-GGATG-3′/5′-CATCC-3′ while the catalytic domain cleaves double-stranded DNA non-specifically at a fixed distance of 9 and 13 nucleotides downstream of the recognition site. FokI exists as an inactive monomer in solution and becomes an active dimmer following the binding to its target DNA and in the presence of some divalent metals. As a functional complex, two molecules of FokI each binding to a double stranded DNA molecule dimerize through the DNA catalytic domain for the effective cleavage of DNA double strands.

ZFN technology has been successfully applied for genetic modification to a variety of organisms, including yeast, plants, fungi and mammals, and even human cell lines. Despite the promise of ZFN technology, however, widespread adoption of this technology is hampered by a bottleneck in custom-engineering zinc fingers capable of high specificity and affinity for the target sites, a process that is labor intensive and associated with high rate of failures. The essence of these endonucleases lies on the DNA binding specificity, which theoretically can be supplanted by any DNA binding proteins/domains when fused with an endonuclease domain, such as a group of TAL effector proteins from bacterial plant pathogens of Xanthomonas.

TAL effectors belong to a large group of bacterial proteins that exist in various strains of Xanthomonas spp. and are translocated into host cells by a type III secretion system, so called type III effectors. Once in host cells, some TAL effectors have been found to transcriptionally activate their corresponding host target genes either for strain virulence (ability to cause disease) or avirulence (capacity to trigger host resistance responses) dependent on the host genetic context. Each effector contains the functional nuclear localization motifs and a potent transcription activation domain that are characteristic of eukaryotic transcription activator. And each effector also contains a central repetitive region consisting of varying numbers of repeat units of 34 amino acids, and the repeat region as DNA binding domain determines the biological specificity of each effector [FIG. 1A]. The repeat is nearly identical except for the variable amino acids at positions 12 and 13, so called repeat variable di-residues (RVD), of each repeat. Recent studies have revealed the recognition of DNA sequences within the promoters of host target genes by the repeat regions of TAL effectors, and the recognition could be simplified in a code that one nucleotide of a target site is corresponding in a sequential order to the RVD of one repeat, with the tandem array of repeats corresponding to a specific, consecutive stretch of DNA. The majority of naturally occurring TAL proteins contain repeat units in a range of 13 to 29 repeats that presumably recognize DNA elements consisting of same number of nucleotides. Furthermore, the so called TAL recognition code could be used to guide the custom-design of novel TAL proteins or repeats with an array of repeat units that can function as DNA binding motifs for a specific and constitutive sequential DNA sequence although such feasibility needs to be determined.

### SUMMARY OF THE INVENTION

Applicants have generated and characterized a TAL nuclease, a hybrid protein derived from FokI and AvrXa7, a member of transcription activator-like (TAL) effector family from phytopathogenic bacteria. The hybrid protein, referred to as TALN, retains both recognition specificity for the target 26-nucleotides of AvrXa7 and the double-stranded DNA cleaving activity of FokI. The TALN cleaves DNA adjacent to the AvrXa7-binding site under optimal conditions in vitro and when expressed promotes the DNA homologous recombination of the LacZ gene that contains the paired target sequences in yeast. Since the modular nature of TAL repeats for target DNA sequences makes it possible to custom-design novel TAL proteins to recognize longer cognate DNA sequence, TAL nucleases represent another tool box of novel enzymes with potential for targeted genome or chromatin modification.

The present invention provides compositions and methods for targeted cleavage of cellular chromatin in a region of interest and/or homologous recombination at a predetermined region of interest in cells. Cells include cultured cells, cells in an organism and cells that have been removed from an organism for treatment in cases where the cells and/or their descendants will be returned to the organism after treatment. A region of interest in cellular chromatin can be, for example, a genomic sequence or portion thereof. Compositions include fusion polypeptides comprising a TAL effector binding domain and a cleavage domain. The cleavage domain can be from any endonuclease. In certain embodiments, the endonuclease is a Type IIS restriction endonuclease. In further embodiments, the Type IIS restriction endonuclease is FokI.

Cellular chromatin can be present in any type of cell including, but not limited to, prokaryotic and eukaryotic cells, fungal cells, plant cells, animal cells, mammalian cells, primate cells and human cells. Cellular chromatin can be present, e.g., in chromosomes or in intracellular genomes of infecting bacteria or viruses.

Thus the invention comprises a method for modifying the genetic material of a cell. The method includes providing a primary cell containing a chromosomal target DNA sequence in which it is desired to have homologous recombination occur; providing a TAL effector endonuclease comprising an endonuclease domain that can cleave double stranded DNA, and a TAL effector domain comprising a plurality of TAL effector repeat sequences that, in combination, bind to a specific nucleotide sequence within the target DNA in the cell; and contacting the target DNA sequence with the TAL effector endonuclease in the cell such that the TAL effector endonuclease cleaves both strands of a nucleotide sequence within or adjacent to the target DNA sequence in the cell. The method can further include providing a nucleic acid comprising a sequence homologous to at least a portion of the target DNA, such that homologous recombination occurs between the target DNA sequence and the nucleic acid. The target DNA sequence can be endogenous to the cell. The cell can be a plant cell or a mammalian cell. The contacting can include transfecting the cell with a vector comprising a TAL effector endonuclease coding sequence, and expressing the TAL effector endonuclease protein in the cell, mechanically injecting a TAL effector endonuclease protein into the cell, delivering a TAL effector endonuclease protein into the cell by means of the bacterial type III secretion system, or introducing a TAL effector endonuclease protein into the cell by electroporation. The endonuclease domain can be from a type III restriction endonuclease (e.g., FokI). The TAL effector domain that binds to a specific nucleotide sequence within the target DNA can include 15 or more DNA binding repeats. The cell can be from an organism selected from the group consisting of a plant, an animal, a mammal, a human, a teleost fish, a fungus, a bacteria or a protozoa.

In another embodiment the invention includes a method for designing a sequence specific TAL effector endonuclease capable of cleaving DNA at a specific location. The method includes identifying a first unique endogenous chromosomal nucleotide sequence adjacent to a second nucleotide sequence at which it is desired to introduce a double-stranded cut; and designing a sequence specific TAL effector endonuclease comprising (a) a plurality of DNA binding repeat domains that, in combination, bind to the first unique endogenous chromosomal nucleotide sequence, and (b) an endonuclease that generates a double-stranded cut at the second nucleotide sequence.

The polarity of the fusion proteins can be such that the TAL effector binding domain is N-terminal to the cleavage domain; alternatively, the cleavage domain can be N-terminal to the TAL effector binding domain. When two fusion proteins of the same polarity are used, their binding sites are on opposite strands of the DNA in the region of interest. In additional embodiments, two fusion proteins of opposite polarity are used. In this case, the binding sites for the two proteins are on the same DNA strand.

In a preferred embodiment of the invention, the cleavage domain is N-terminal to the TAL sequence. While both orientations of each fusion (FN-TAL, TAL-FN) are functional as demonstrated herein, the polarity of FN-TAL is preferred as the transcription activation domain at the C-terminal end is intact and retains the transcription activator activity which enables one to measure the DNA binding specificity of naturally occurring TAL or newly engineered TAL used for nuclease fusion. Also, this orientation may give the flexibility of spacer lengths between two target sites and the orientation of target sites by themselves when designing TALNs. For example, FN-TAL works for 30 nt between two sites, while TAL-FN works for 19 nt in our experiments. This is important when designing TALs in considering target sites, spacer lengths and the like.

According to the invention, the fusion protein can be expressed in a cell, e.g., by delivering the fusion protein to the cell or by delivering a polynucleotide encoding the fusion protein to a cell, wherein the polynucleotide, if DNA, is transcribed, and an RNA molecule delivered to the cell or a transcript of a DNA molecule delivered to the cell is translated, to generate the fusion protein. Methods for polynucleotide and polypeptide delivery to cells are known in the art and are presented elsewhere in this disclosure.

Targeted mutations resulting from the aforementioned method include, but are not limited to, point mutations (i.e., conversion of a single base pair to a different base pair), substitutions (i.e., conversion of a plurality of base pairs to a different sequence of identical length), insertions or one or more base pairs, deletions of one or more base pairs and any combination of the aforementioned sequence alterations.

Methods for targeted recombination (for, e.g., alteration or replacement of a sequence in a chromosome or a region of interest in cellular chromatin) are also provided. For example, a mutant genomic sequence can be replaced by a wild-type sequence, e.g., for treatment of genetic disease or inherited disorders. In addition, a wild-type genomic sequence can be replaced by a mutant sequence, e.g., to prevent function of an oncogene product or a product of a gene involved in an inappropriate inflammatory response. Furthermore, one allele of a gene can be replaced by a different allele.

The invention also includes a TAL effector endonuclease comprising an endonuclease domain and a TAL effector DNA binding domain specific for a particular DNA sequence. The TAL effector endonuclease can further include a purification tag. The endonuclease domain can be from a type III restriction endonuclease (e.g., FokI).

### DESCRIPTION OF THE FIGURES

The patent or application file contains at least one drawing executed in color. Copies of this patent or patent application publication with color drawing(s) will be provided by the Office upon request and payment of the necessary fee.

FIG. 1. Schematic of TAL effector AvrXa7 and its target DNA sequence. (A) A typical TAL effector contains a central region of 34 or 35 amino acid direct repeats (open boxes) and three nuclear localization motifs (NLS, black thick line) as well as a transcription activation domain (AD, red solid box) at the C-terminus. The representative 34 amino acid repeat is shown below with the variable amino acid residues at the position 12 and 13 in red and shaded in gray. (B) AvrXa7 contains a 288 amino acid (aa) N-terminal region, the central 26 tandem repeats (shown as box) of 34 amino acid residues and a C-terminal portion of 286 amino acids. The repeat is highly conserved, except for residues at positions 12 and 13 (shown within each repeat; N, asparagine; I, isoleucine; H, histidine; G, glycine; S, serine; D, aspartic acid; *, missing residue at 13 position). The binding specificity of AvrXa7 to Os11N3 is defined by the diamino-acids in the repeat unit with nucleotides (T, thymine; A, adenine; C, cytosine; G, Guanosine) in the DNA target.

FIG. 2. Binding specificity of AvrXa7-FokI fusion protein to its target DNA. (A) Schematic of the fused full-length AvrXa7 and FokI cleavage domain (FN). (B) Transient activation of Os11N3 promoter with the reporter gene GFP (UPTOs11N3::GFP) by avrXa7 (35S::avrXa7), the chimeric gene avrXa7-FokI (35S::avrXa7-FokI) or lack thereof (control) when expressed under the cauliflower mosaic virus 35S promoter in leaves of Nicotiana benthamiana.

FIG. 3. Expression of AvrXa7-FokI fusion protein. (A) The coomassie blue stained SDS-PAGE gel image of AvrXa7-FokI. Lane 1, marker proteins; lane 2, cell lysate without IPTG induction; lane 3, IPTG induction for 3 hr; lane 4, extraction through Ni-chromtography purification; lane 5, extract through gel infiltration from extraction in lane 4. (B) Western blot analysis of AvrXa7-FokI. The identical samples in (A) were probed with anti-FLAG antiserium.

FIG. 4. Binding specificity of AvrXa7-FokI fusion protein to its target DNA. (A) Sequence of oligonucleotides used in the electrophoresis mobility shift (EMS) assay. (B) EMS assay for DNA binding specificity of AvrXa7-FokI to Os11N3 target site. AvrXa7-FokI binds preferentially to Os11N3 target element but not to the mutated version (left panel). The binding of labeled Os11N3 probe by AvrXa7-FokI is effectively competed by the excess amount of cold Os11N3 oligonucleotides (middle three lanes under Os11N3) but not by the cold mutated Os11N3 DNA (right panel under Os11N3M). Positions of the bound and free probes are indicated on the left.

FIG. 5. DNA digestion with AvrXa7-FokI. (A) Schematic of linearized plasmids used in digestion reactions. The plasmid DNA was linearized with EcoNI. pTOP/Os11N3 represents a 400 bp promoter region including the 5′-UTR of Os11N3 (open box) in pTOPO cloning vector. The AvrXa7 binding sequence for wild type (1) and mutation (2) under the open box is underlined and in red. The numbers (2129 bp and 2971 bp) indicate the positions of nucleotides relative to EcoN1 site at left side. pTOP/GFP presents GFP coding sequence cloned into pTOPO vector. (B) Gel image of EcoNI linearized plasmid DNA in (A) with AvrXa7-FokI. M, 1 kb marker, 1, pTOP/Os11N3 wild type (1); 2, mutated pTOP/Os11N3 (2); and 3, pTOP/GFP. (C) Same amount of linearized pTOP/Os11N3 was digested with increasing amount of AvrXa7-FokI for 1 hour at 37° C. The expected fragment sizes are indicated at the right. Unsaturated AvrXa7-FokI digestion of pTOP/Os11N3 in different length of digestion time in hours (above each lane).

FIG. 6. DNA sequencing reveals the cleavage sites of cognate DNA by AvrXa7-FokI. (A) Interpretive cleavage sites of ds DNA by AvrXa7-FokI. M13F and M13R are primers used for sequencing fragments at the left and right side of binding sequence (boxed in yellow shade), respectively. The red arrow head indicates the cleavage site of upper strand; the single and double dark arrow head denote the two obvious cleavage sites of lower DNA strand. The sequence chromatogram in (A) depicts the DNA fragment (0.8 kb) downstream the cleavage site. The chromatogram, which represents the upper strand sequence around the cleavage site in (A), is reverse-complemented for ease of viewing. The region delimited by the vertical dash line appears to be the AvrXa7-FokI binding site whose correct sequence is boxed in yellow shade. (B) The chromatogram represents the lower strand sequence of DNA fragment left the cleavage site. The dark arrow heads indicate the prominent cleavage sites corresponding to those in (A). The dash line delimits the AvrXa7-FokI binding site.

FIG. 7. Yeast SSA assay to detect FN-AvrXa7 induced homologous recombination. (A) Schematic of the reporter constructs (drawn not in scale) with AvrXa7 EBE sites. Two nonfunctional LacZ gene fragments (LacZn and LacZc, blue solid bar) were separated the DNA fragment of URA3 gene (gray line) and a multiple cloning site (black line). The two duplicated LacZ coding sequences are hatched blue boxes. The reporter constructs are designated as pS (single EBE site), pDH (double sites in a head-to-head orientation separated by the red-lined spacers) followed by the numbers of spacer nucleotides. HR denotes homologous recombination, “−” denotes low β-galactosidase activity indicative of no HR; “+” is for increased β-galactosidase activity, while “++” for higher frequency of HR. (B) The β-galactosidase activities from each reporter plasmid in (A) are presented in graph.

FIG. 8. DNA and amino acid sequence of FN-AvrXa7 (1, 2) and AvrXa7-FN (3, 4). 1. Bold sequence corresponds to the FokI nuclease domain. The open reading frame of AvrXa7 is defined by red colored ATG and TAG. Restriction sites BglII and SpeI used for cloning are underlined. 2. The sequence of FokI nuclease domain is bolded. The N-terminal and C-terminal sequences of AvrXa7 are underlined. The first 34 amino acid repeat is shade in gray. The repeat variable di-residue (RVD) amino acids of each repeat are in red.

FIG. 9. Yeast SSA assay for FN-AvrXa7 stimulated HR. (A) The sense strand DNA sequences of the AvrXa7 EBE sites in reporter constructs used for FN-AvrXa7 nuclease activity. The EBE sites are red capital letters. The restriction sites BglII and SpeI used for cloning are underlined. The spacer DNA sequences between the two EBE sites are in lower case. (B) The colony-lift filter assay for yeast cells containing the reporter (labels on left side) and effector constructs (above the first panel; Vector, plasmid lacking FN-AvrXa7 and FN-AvrXa7, plasmid with FN-AvrXa7. The filters were photographed 5 hrs after stained with X-gal in Z-buffer.

FIG. 10. (A) Schematics of yeast URA3 gene in chromosome 5 (ChrV) with the integrated targeted sequences in frame with the ORF of URA3 gene. The target sites are underlined with the spacer sequence in lower case letters. The ZFNs and TALNs bind to the target sites and the FokI nuclease domains (FN) dimerize and cleave double stranded DNA between the target sites. (B) Genomic DNA sequences at the sites of mutations induced by ZFNs. Parental strain (PT) and five representatives of mutants (M) with insertion (red lower case letter) and deletions (red dashes) were shown. (C) Genomic sequences at the sites of mutations caused by TALNs. The lower case letters in red indicate insertions and the dashed lines denote DNA sequences deleted in the mutants (M) compared to the parental strain (PT).

FIG. 11. (A) Four modules each encoding 34 AA with the twelfth and thirteenth residues (RVD) that specifically recognize one of the four nucleotides (i.e., NI for A, NG for T, NN for G, and HD for C, respectively). Each module consists of two halves of adjacent repeats (2nd half in bold). The 4 base pair overhangs (XXXX) at each end are generated by BsmBI whose recognition site is GAGACG (underlined). The 4 bp overhangs are compatible with the overhangs of adjacent repeat units on either side—thus allowing sequential assembly of the 102 bp repeats and the resulting TAL effector match an array of specific nucleotides in the target gene. Dots denote nucleotides or amino acids not shown. (B) Two EBE sites at positions +16 and +597 (relative to the “A” of the ATG start codon) of the yeast URA3 gene (region delimited by red typeface ATG and TTA) on chromosome 5 (ChrV) chosen as target sites (boxed sequences underlined) for engineering TALNs (TalU1-L and TalU1-R for the EBE site beginning at +16 and TalU2-L and TalU2-R for the position at +597). (C) The RVD sequences of the four TALNs (TalU1-L, —R, and TalU2-L, —R) and their corresponding recognition DNA sequences are shown with the sequential order of repeats that were custom-synthesized using the individual modules illustrated in (A). (D) and (E) DNA alignment of URA3 alleles retrieved from the parental strain (WT) and its derivative mutants (ura3-1, -2, -3, -4, -5, -10, -11 and -12) with insertions (red letters)/deletions (dashes in red) relevant to two sets of TALNs (TalU1-L, —R and TalU2-L, —R). The dual TALN target sites (TalU1 EBE and TalU2 EBE) are underlined.

FIG. 12. Target sites of the eGFP gene by TALNs and design of TALE endonuclease.

FIG. 13. GFP expression in the presence of increasing amounts of eGFP dTALEN of transfected human HEK239T cells with the EGFP expression plasmid.

FIG. 14. Quantification of GFP-transfected cells by FACS. 50,000 cells from each treatment group were analyzed by FACS for GFP expression.

FIG. 15. The GFP gene was amplified and sequenced from treated cells. FIG. 15 shows the sequence used for design of the primers.

FIG. 16. Targeted disruption of the GFP gene was observed. GFP-TAL1 (4 clones); 0 mg TALEN transfected; No insertions/deletions. GFP-TAL2 (10 clones); 0.5 mg/well TALEN (0.5 ug/well); 5/10 clones contain deletions at target site. Sequences from the cells are given.

### DETAILED DESCRIPTION OF THE INVENTION

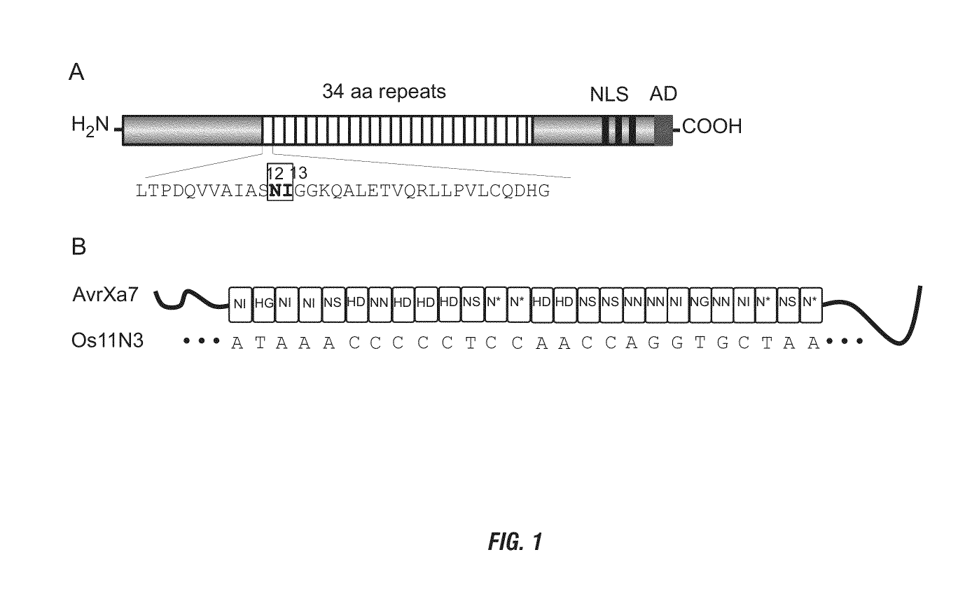

AvrXa7 is a TAL type III effector from Xanthomonas oryzae pv. oryzae (Xoo), the causal pathogen of bacterial blight of rice. It contains a unique combination of RVDs of 26 repeats (FIG. 1B). For some Xoo strains, AvrXa7 is a key virulence factor in susceptible rice, whereas it is also an avirulence determinant in the otherwise resistant plant containing the cognate resistance gene Xa7. As the essential virulence factor, AvrXa7 activates the rice gene Os11N3 to induce a state of disease susceptibility. The gene induction by AvrXa7 is mediated through its recognition of the DNA element within the promoter region of Os11N3, an element we refer to here as effector binding element (EBE) (sequence shown in FIG. 1B). As the proof-of-principle, we tested the feasibility of generating a new type of endonucleases by utilizing the sequence specificity of AvrXa7 and the nuclease catalytic activity of the endonuclease FokI. Applicants have created a TALN by fusing the full-length AvrXa7 to the FN and characterization of its nuclease activity in vitro and in a yeast assay.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### DEFINITIONS

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “TAL effector DNA binding protein” (or binding domain) or a “TAL effector DNA recognition sequence” is a protein encompassing a series of repeat variable-diresidues (RVDs) within a larger protein, that binds DNA in a sequence-specific manner. The RVD regions of TAL effectors are polymorphisms within TALs typically at positions 12 and 13 in repeating units of typically 34 amino acids that bind for specific nucleotides and together with a plurality of repeating unit intervals make up the specific TAL effector DNA binding domain.

TAL effector DNA binding protein domains (their RVDs) can be “engineered” to bind to a predetermined nucleotide sequence. Non-limiting examples of methods for engineering the same are design and selection. A designed TAL effector DNA binding protein is a protein not occurring in nature whose design/composition results principally from rational criteria. Rational criteria for design include application of substitution rules and computerized algorithms for processing information in a database storing information of existing RVD designs and binding data.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value there between or thereabove), preferably between about 100 and 1,000 nucleotides in length (or any integer there between), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination there between, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage domain” comprises one or more polypeptide sequences which possesses catalytic activity for DNA cleavage. A cleavage domain can be contained in a single polypeptide chain or cleavage activity can result from the association of two (or more) polypeptides.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a TAL effector sequence DNA-binding domain and a cleavage domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression.

“Eucaryotic” cells include, but are not limited to, fungal cells (such as yeast), plant cells, animal cells, mammalian cells and human cells.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a TAL effector DNA-binding domain is fused to a cleavage domain, the TAL effector DNA-binding domain and the cleavage domain are in operative linkage if, in the fusion polypeptide, the TAL effector DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

### Target Sites

The disclosed methods and compositions include fusion proteins comprising a cleavage domain and a TAL effector DNA binding domain, or DNA recognition sequence in which the RVDs, by binding to a sequence in cellular chromatin (e.g., a target site or a binding site), directs the activity of the cleavage domain (or cleavage half-domain) to the vicinity of the sequence and, hence, induces cleavage in the vicinity of the target sequence. As set forth elsewhere in this disclosure, particular RVDs within a TAL binding domain can be engineered to bind to virtually any desired sequence. Accordingly, after identifying a region of interest containing a sequence at which cleavage or recombination is desired, one or more TAL effector DNA binding domains can be engineered to bind to one or more sequences in the region of interest. Expression of a fusion protein comprising a TAL effector DNA binding domain and a cleavage domain, in a cell, effects cleavage in the region of interest.

Selection of a sequence in cellular chromatin for binding by a TAL effector binding domain (e.g., a target site) can be accomplished, by any method known to those of skill in the art. For example simple visual inspection of a nucleotide sequence can be used for selection of a target site. Accordingly, any means for target site selection can be used in the claimed methods.

### Sequence-Specific Endonucleases

Sequence-specific nucleases and recombinant nucleic acids encoding the sequence-specific endonucleases are provided herein. The sequence-specific endonucleases can include TAL effector DNA binding domains and endonuclease domains. Thus, nucleic acids encoding such sequence-specific endonucleases can include a nucleotide sequence from a sequence-specific TAL effector linked to a nucleotide sequence from a nuclease.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Because the relationship between the TAL amino acid sequence and the target binding site is simple, target sites can be predicted for TAL effectors, and TAL effectors also can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type III restriction endonuclease such as FokI (Kim et al. (1996) Proc. Natl. Acad. Sci. USA 93:1156-1160). Other useful endonucleases may include, for example, HhaI, HindIII, NotI, BbvC1, EcoRI, BglI, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TAL effector endonuclease as provided herein can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TAL effector endonuclease can be engineered to target a particular cellular sequence. A nucleotide sequence encoding the desired TAL effector endonuclease can be inserted into any suitable expression vector, and can be linked to one or more expression control sequences. For example, a nuclease coding sequence can be operably linked to a promoter sequence that will lead to constitutive expression of the endonuclease in the species of plant to be transformed. Alternatively, an endonuclease coding sequence can be operably linked to a promoter sequence that will lead to conditional expression (e.g., expression under certain nutritional conditions).

### Cleavage Domains

The cleavage domain portion of the fusion proteins disclosed herein can be obtained from any endo- or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31, 982. Thus, in one embodiment, fusion proteins comprise the cleavage domain (or cleavage half-domain) from at least one Type IIS restriction enzyme.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95: 10,570-10,575. Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using TAL-FokI fusions, two fusion proteins, each comprising a FokI cleavage half-domain, can be used to reconstitute a catalytically active cleavage domain. Parameters for targeted cleavage and targeted sequence alteration using TAL-FokI fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Additional restriction enzymes also contain separable binding and cleavage domains, and these are contemplated by the present disclosure. See, for example, Roberts et al. (2003) Nucleic Acids Res. 31:418-420. Examples of Type IIS Restriction Enzymes include: Aar I, BsrB I, SspD5 I, Ace III, BsrD I, Sth132 I, Aci I, BstF5 I, Sts I, Alo I, Btr I, TspDT I, Bae I, Bts I, TspGW I, Bbr7 I, Cdi I, Tth111 II, Bbv I, CjeP I, UbaP I, Bbv II, Drd II, Bsa I, BbvC I, Eci I, BsmB I, Bcc I, Eco31 I, Bce83 I, Eco57 I, BceA I, Eco57M I, Bcef I, Esp3 I, Bcg I, Fau I, BciV I, Fin I, Bfi I, Fok I, Bin I, Gdi II, Bmg I, Gsu I, Bpu10 I, Hga I, BsaX I, Hin4 II, Bsb I, Hph I, BscA I, Ksp632 I, BscG I, Mbo II, BseR I, Mly I, BseY I, Mme I, Bsi I, Mnl I, Bsm I, Pfl1108 I, BsmA I, Ple I, BsmF I, Ppi I, Bsp24 I, Psr I, BspG I, RleA I, BspM I, Sap I, BspNC I, SfaN I, Bsr I, and Sim I.

### TAL Effector DNA Domain-Cleavage Domain Fusions

Methods for design and construction of fusion proteins (and polynucleotides encoding same) are known to those of skill in the art. For example, methods for the design and construction of fusion protein comprising TAL proteins (and polynucleotides encoding same) are described in U.S. Pat. Nos. 6,453,242 and 6,534,261. In certain embodiments, polynucleotides encoding such fusion proteins are constructed. These polynucleotides can be inserted into a vector and the vector can be introduced into a cell (see below for additional disclosure regarding vectors and methods for introducing polynucleotides into cells).

In certain embodiments of the methods described herein, a fusion protein comprises a TAL effector binding domain from AvrXa7 and a cleavage half-domain from the FokI restriction enzyme, and two such fusion proteins are expressed in a cell. Expression of two fusion proteins in a cell can result from delivery of the two proteins to the cell; delivery of one protein and one nucleic acid encoding one of the proteins to the cell; delivery of two nucleic acids, each encoding one of the proteins, to the cell; or by delivery of a single nucleic acid, encoding both proteins, to the cell. In additional embodiments, a fusion protein comprises a single polypeptide chain comprising two cleavage half domains and a TAL AvrXa7 binding domain. In this case, a single fusion protein is expressed in a cell and, without wishing to be bound by theory, is believed to cleave DNA as a result of formation of an intramolecular dimer of the cleavage half-domains.

In certain embodiments, the components of the fusion proteins (e.g, TAL-FokI fusions) are arranged such that the cleavage domain is nearest the amino terminus of the fusion protein, and the TAL domain is nearest the carboxy-terminus. This provides certain advantages such as the retention of the transcription activator activity which enables one to measure the DNA binding specificity of naturally occurring TAL or newly engineered TAL used for nuclease fusion and this orientation may give the flexibility of spacer lengths.

### Methods for Targeted Cleavage

The disclosed methods and compositions can be used to cleave DNA at a region of interest in cellular chromatin (e.g., at a desired or predetermined site in a genome, for example, in a gene, either mutant or wild-type). For such targeted DNA cleavage, TAL binding domain is engineered to bind a target site at or near the predetermined cleavage site, and a fusion protein comprising the engineered TAL binding domain and a cleavage domain is expressed in a cell. Upon binding of the TAL RVDs portion of the fusion protein to the target site, the DNA is cleaved near the target site by the cleavage domain.

For targeted cleavage using a TAL binding domain-cleavage domain fusion polypeptide, the binding site can encompass the cleavage site, or the near edge of the binding site can be 1, 2, 3, 4, 5, 6, 10, 25, 50 or more nucleotides (or any integral value between 1 and 50 nucleotides) from the cleavage site. The exact location of the binding site, with respect to the cleavage site, will depend upon the particular cleavage domain, and the length of any linker.

Thus, the methods described herein can employ an engineered TAL effector DNA binding domain fused to a cleavage domain. In these cases, the binding domain is engineered to bind to a target sequence, at or near which cleavage is desired. The fusion protein, or a polynucleotide encoding same, is introduced into a cell. Once introduced into, or expressed in, the cell, the fusion protein binds to the target sequence and cleaves at or near the target sequence. The exact site of cleavage depends on the nature of the cleavage domain and/or the presence and/or nature of linker sequences between the binding and cleavage domains. Optimal levels of cleavage can also depend on both the distance between the binding sites of the two fusion proteins (See, for example, Smith et al. (2000) Nucleic Acids Res. 28:3361-3369; Bibikova et al. (2001) Mol. Cell. Biol. 21:289-297) and the length of the ZC linker in each fusion protein.

In certain embodiments, the cleavage domain comprises two cleavage half-domains, both of which are part of a single polypeptide comprising a binding domain, a first cleavage half-domain and a second cleavage half-domain. The cleavage half-domains can have the same amino acid sequence or different amino acid sequences, so long as they function to cleave the DNA.

Cleavage half-domains may also be provided in separate molecules. For example, two fusion polypeptides may be introduced into a cell, wherein each polypeptide comprises a binding domain and a cleavage half-domain. The cleavage half-domains can have the same amino acid sequence or different amino acid sequences, so long as they function to cleave the DNA. Further, the binding domains bind to target sequences which are typically disposed in such a way that, upon binding of the fusion polypeptides, the two cleavage half-domains are presented in a spatial orientation to each other that allows reconstitution of a cleavage domain (e.g., by dimerization of the half-domains), thereby positioning the half-domains relative to each other to form a functional cleavage domain, resulting in cleavage of cellular chromatin in a region of interest. Generally, cleavage by the reconstituted cleavage domain occurs at a site located between the two target sequences. One or both of the proteins can be engineered to bind to its target site.

The two fusion proteins can bind in the region of interest in the same or opposite polarity, and their binding sites (i.e., target sites) can be separated by any number of nucleotides, e.g., from 0 to 200 nucleotides or any integral value therebetween. In certain embodiments, the binding sites for two fusion proteins, each comprising a TAL effector binding domain and a cleavage half-domain, can be located between 5 and 18 nucleotides apart, for example, 5-8 nucleotides apart, or 15-18 nucleotides apart, or 6 nucleotides apart, or 16 nucleotides apart, as measured from the edge of each binding site nearest the other binding site, and cleavage occurs between the binding sites.

The site at which the DNA is cleaved generally lies between the binding sites for the two fusion proteins. Double-strand breakage of DNA often results from two single-strand breaks, or “nicks,” offset by 1, 2, 3, 4, 5, 6 or more nucleotides, (for example, cleavage of double-stranded DNA by native FokI results from single-strand breaks offset by 4 nucleotides). Thus, cleavage does not necessarily occur at exactly opposite sites on each DNA strand. In addition, the structure of the fusion proteins and the distance between the target sites can influence whether cleavage occurs adjacent a single nucleotide pair, or whether cleavage occurs at several sites. However, for many applications, including targeted recombination and targeted mutagenesis (see infra) cleavage within a range of nucleotides is generally sufficient, and cleavage between particular base pairs is not required.

As noted above, the fusion protein(s) can be introduced as polypeptides and/or polynucleotides. For example, two polynucleotides, each comprising sequences encoding one of the aforementioned polypeptides, can be introduced into a cell, and when the polypeptides are expressed and each binds to its target sequence, cleavage occurs at or near the target sequence. Alternatively, a single polynucleotide comprising sequences encoding both fusion polypeptides is introduced into a cell. Polynucleotides can be DNA, RNA or any modified forms or analogues or DNA and/or RNA.

To enhance cleavage specificity, additional compositions may also be employed in the methods described herein. For example, single cleavage half-domains can exhibit limited double-stranded cleavage activity. In methods in which two fusion proteins are introduced into the cell, either protein specifies an approximately 9-nucleotide target site. Although the aggregate target sequence of 18 nucleotides is likely to be unique in a mammalian genome, any given 9-nucleotide target site occurs, on average, approximately 23,000 times in the human genome. Thus, non-specific cleavage, due to the site-specific binding of a single half-domain, may occur. Accordingly, the methods described herein contemplate the use of a dominant-negative mutant of a cleavage half-domain such as FokI (or a nucleic acid encoding same) that is expressed in a cell along with the two fusion proteins. The dominant-negative mutant is capable of dimerizing but is unable to cleave, and also blocks the cleavage activity of a half-domain to which it is dimerized. By providing the dominant-negative mutant in molar excess to the fusion proteins, only regions in which both fusion proteins are bound will have a high enough local concentration of functional cleavage half-domains for dimerization and cleavage to occur. At sites where only one of the two fusion proteins is bound, its cleavage half-domain forms a dimer with the dominant negative mutant half-domain, and undesirable, non-specific cleavage does not occur.

Three catalytic amino acid residues in the FokI cleavage half-domain have been identified: Asp 450, Asp 467 and Lys 469. Bitinaite et al. (1998) Proc. Natl. Acad. Sci. USA 95: 10,570-10,575. Thus, one or more mutations at one of these residues can be used to generate a dominant negative mutation. Further, many of the catalytic amino acid residues of other Type IIS endonucleases are known and/or can be determined, for example, by alignment with FokI sequences and/or by generation and testing of mutants for catalytic activity.

In addition to the fusion molecules described herein, targeted replacement of a selected genomic sequence also requires the introduction of the replacement (or donor) sequence. The donor sequence can be introduced into the cell prior to, concurrently with, or subsequent to, expression of the fusion protein(s). The donor polynucleotide contains sufficient homology to a genomic sequence to support homologous recombination between it and the genomic sequence to which it bears homology. Approximately 25, 50, 100 or 200 nucleotides or more of sequence homology between a donor and a genomic sequence (or any integral value between 10 and 200 nucleotides, or more) will support homologous recombination therebetween. Donor sequences can range in length from 10 to 5,000 nucleotides (or any integral value of nucleotides therebetween) or longer. It will be readily apparent that the donor sequence is typically not identical to the genomic sequence that it replaces. For example, the sequence of the donor polynucleotide can contain one or more single base changes, insertions, deletions, inversions or rearrangements with respect to the genomic sequence, so long as sufficient homology is present to support homologous recombination. Alternatively, a donor sequence can contain a non-homologous sequence flanked by two regions of homology. Additionally, donor sequences can comprise a vector molecule containing sequences that are not homologous to the region of interest in cellular chromatin. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

To simplify assays (e.g., hybridization, PCR, restriction enzyme digestion) for determining successful insertion of the donor sequence, certain sequence differences may be present in the donor sequence as compared to the genomic sequence. Preferably, if located in a coding region, such nucleotide sequence differences will not change the amino acid sequence, or will make silent amino acid changes (i.e., changes which do not affect the structure or function of the protein). The donor polynucleotide can optionally contain changes in sequences corresponding to the TAL effector domain binding (or recognition) sites in the region of interest, to prevent cleavage of donor sequences that have been introduced into cellular chromatin by homologous recombination.

The donor polynucleotide can be DNA or RNA, single-stranded or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV).

Without being bound by one theory, it appears that the presence of a double-stranded break in a cellular sequence, coupled with the presence of an exogenous DNA molecule having homology to a region adjacent to or surrounding the break, activates cellular mechanisms which repair the break by transfer of sequence information from the donor molecule into the cellular (e.g., genomic or chromosomal) sequence; i.e., by a processes of homologous recombination. Applicants' methods advantageously combine the powerful targeting capabilities of engineered TALs with a cleavage domain (or cleavage half-domain) to specifically target a double-stranded break to the region of the genome at which recombination is desired.

For alteration of a chromosomal sequence, it is not necessary for the entire sequence of the donor to be copied into the chromosome, as long as enough of the donor sequence is copied to effect the desired sequence alteration.

In certain embodiments, a homologous chromosome can serve as the donor polynucleotide. Thus, for example, correction of a mutation in a heterozygote can be achieved by engineering fusion proteins which bind to and cleave the mutant sequence on one chromosome, but do not cleave the wild-type sequence on the homologous chromosome. The double-stranded break on the mutation-bearing chromosome stimulates a homology-based “gene conversion” process in which the wild-type sequence from the homologous chromosome is copied into the cleaved chromosome, thus restoring two copies of the wild-type sequence.

Further increases in efficiency of targeted recombination, in cells comprising fusion molecule and a donor DNA molecule, are achieved by blocking the cells in the G₂ phase of the cell cycle, when homology-driven repair processes are maximally active. Such arrest can be achieved in a number of ways. For example, cells can be treated with e.g., drugs, compounds and/or small molecules which influence cell-cycle progression so as to arrest cells in G₂ phase. Exemplary molecules of this type include, but are not limited to, compounds which affect microtubule polymerization (e.g., vinblastine, nocodazole, Taxol), compounds that interact with DNA (e.g., cis-platinum(II) diamine dichloride, Cisplatin, doxorubicin) and/or compounds that affect DNA synthesis (e.g., thymidine, hydroxyurea, L-mimosine, etoposide, 5-fluorouracil). Additional increases in recombination efficiency are achieved by the use of histone deacetylase (HDAC) inhibitors (e.g., sodium butyrate, trichostatin A) which alter chromatin structure to make genomic DNA more accessible to the cellular recombination machinery.

Additional methods for cell-cycle arrest include overexpression of proteins which inhibit the activity of the CDK cell-cycle kinases, for example, by introducing a cDNA encoding the protein into the cell or by introducing into the cell an engineered ZFP which activates expression of the gene encoding the protein. Cell-cycle arrest is also achieved by inhibiting the activity of cyclins and CDKs, for example, using RNAi methods (e.g., U.S. Pat. No. 6,506,559) or by introducing into the cell an engineered ZFP which represses expression of one or more genes involved in cell-cycle progression such as, for example, cyclin and/or CDK genes. See, e.g., U.S. Pat. No. 6,534,261 for methods for the synthesis of engineered TAL proteins for regulation of gene expression.

Methods to Screen for Cellular Factors that Facilitate Homologous Recombination

Since homologous recombination is a multi-step process requiring the modification of DNA ends and the recruitment of several cellular factors into a protein complex, the addition of one or more exogenous factors, along with donor DNA and vectors encoding TAL-cleavage domain fusions, can be used to facilitate targeted homologous recombination. An exemplary method for identifying such a factor or factors employs analyses of gene expression using microarrays (e.g., Affymetrix Gene Chip® arrays) to compare the mRNA expression patterns of different cells. For example, cells that exhibit a higher capacity to stimulate double strand break-driven homologous recombination in the presence of donor DNA and TAL-cleavage domain fusions, either unaided or under conditions known to increase the level of gene correction, can be analyzed for their gene expression patterns compared to cells that lack such capacity. Genes that are upregulated or downregulated in a manner that directly correlates with increased levels of homologous recombination are thereby identified and can be cloned into any one of a number of expression vectors. These expression constructs can be co-transfected along with TAL-cleavage domain fusions and donor constructs to yield improved methods for achieving high-efficiency homologous recombination.

### Expression Vectors

A nucleic acid encoding one or more fusion proteins can be cloned into a vector for transformation into prokaryotic or eukaryotic cells for replication and/or expression. Vectors can be prokaryotic vectors, e.g., plasmids, or shuttle vectors, insect vectors, or eukaryotic vectors. A nucleic acid encoding a TAL effector binding domain can also be cloned into an expression vector, for administration to a plant cell, animal cell, preferably a mammalian cell or a human cell, fungal cell, bacterial cell, or protozoal cell.

To obtain expression of a cloned gene or nucleic acid, sequences encoding a fusion protein are typically subcloned into an expression vector that contains a promoter to direct transcription.

Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The choice of promoters to be included depends upon several factors, including, but not limited to, efficiency, selectability, inducibility, desired expression level, and cell or tissue specificity. For example, tissue-, organ- and cell-specific promoters that confer transcription only or predominantly in a particular tissue, organ, and cell type, respectively, can be used. In some embodiments, promoters specific to vegetative tissues such as the stem, parenchyma, ground meristem, vascular bundle, cambium, phloem, cortex, shoot apical meristem, lateral shoot meristem, root apical meristem, lateral root meristem, leaf primordium, leaf mesophyll, or leaf epidermis can be suitable regulatory regions. In some embodiments, promoters that are essentially specific to seeds (“seed-preferential promoters”) can be useful. Seed-specific promoters can promote transcription of an operably linked nucleic acid in endosperm and cotyledon tissue during seed development. Alternatively, constitutive promoters can promote transcription of an operably linked nucleic acid in most or all tissues of a plant, throughout plant development. Other classes of promoters include, but are not limited to, inducible promoters, such as promoters that confer transcription in response to external stimuli such as chemical agents, developmental stimuli, or environmental stimuli.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Non-limiting examples of promoters that can be included in the nucleic acid constructs provided herein include the cauliflower mosaic virus (CaMV) 35S transcription initiation region, the 1′ or 2′ promoters derived from T-DNA of Agrobacterium tumefaciens, promoters from a maize leaf-specific gene described by Busk ((1997) Plant J 11:1285-1295), knl-related genes from maize and other species, and transcription initiation regions from various plant genes such as the maize ubiquitin-1 promoter.

A 5′ untranslated region (UTR) is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences. A polyadenylation region at the 3′-end of a coding region can also be operably linked to a coding sequence. The polyadenylation region can be derived from the natural gene, from various other plant genes, or from an Agrobacterium T-DNA.

The vectors provided herein also can include, for example, origins of replication, and/or scaffold attachment regions (SARs). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag” tag (Kodak, New Haven, Conn.) sequences typically are expressed as a fusion with the encoded polypeptide. Such tags can be inserted anywhere within the polypeptide, including at either the carboxyl or amino terminus.

It will be understood that more than one regulatory region may be present in a recombinant polynucleotide, e.g., introns, enhancers, upstream activation regions, and inducible elements.

Recombinant nucleic acid constructs can include a polynucleotide sequence inserted into a vector suitable for transformation of cells (e.g., plant cells or animal cells). Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.).

Suitable bacterial and eukaryotic promoters are well known in the art and described, e.g., in Sambrook et al., Molecular Cloning, A Laboratory Manual (2nd ed. 1989; 3rd ed., 2001); Kriegler, Gene Transfer and Expression: A Laboratory Manual (1990); and Current Protocols in Molecular Biology (Ausubel et al., supra. Bacterial expression systems for expressing the ZFP are available in, e.g., E. coli, Bacillus sp., and Salmonella (Palva et al., Gene 22:229-235 (1983)). Kits for such expression systems are commercially available. Eukaryotic expression systems for mammalian cells, yeast, and insect cells are well known by those of skill in the art and are also commercially available.

The promoter used to direct expression of a TAL-cleavage domain fusion protein-encoding nucleic acid depends on the particular application. For example, a strong constitutive promoter is typically used for expression and purification of TAL-cleavage domain fusion proteins. In contrast, when a TAL-cleavage domain fusion protein is administered in vivo for gene regulation, either a constitutive or an inducible promoter is used, depending on the particular use of the TAL-cleavage domain fusion protein. In addition, a preferred promoter for administration of a TAL-cleavage domain fusion protein can be a weak promoter, such as HSV TK or a promoter having similar activity. The promoter typically can also include elements that are responsive to transactivation, e.g., hypoxia response elements, Ga14 response elements, lac repressor response element, and small molecule control systems such as tet-regulated systems and the RU-486 system (see, e.g., Gossen & Bujard, PNAS 89:5547 (1992); Oligino et al., Gene Ther. 5:491-496 (1998); Wang et al., Gene Ther. 4:432-441 (1997); Neering et al., Blood 88:1147-1155 (1996); and Rendahl et al., Nat. Biotechnol. 16:757-761 (1998)). The MNDU3 promoter can also be used, and is preferentially active in CD34+ hematopoietic stem cells.

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to a nucleic acid sequence encoding the TAL-cleavage domain fusion protein and signals required, e.g., for efficient polyadenylation of the transcript, transcriptional termination, ribosome binding sites, or translation termination. Additional elements of the cassette may include, e.g., enhancers, and heterologous splicing signals.

The particular expression vector used to transport the genetic information into the cell is selected with regard to the intended use of the TAL-cleavage domain fusion protein, e.g., expression in plants, animals, bacteria, fungus, protozoa, etc. (see expression vectors described below). Standard bacterial expression vectors include plasmids such as pBR322-based plasmids, pSKF, pET23D, and commercially available fusion expression systems such as GST and LacZ. An exemplary fusion protein is the maltose binding protein, “MBP.” Such fusion proteins are used for purification of the TAL-cleavage domain fusion protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Ban virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with a TAL-cleavage domain fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce plant, bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, ultrasonic methods (e.g., sonoporation), liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

### Nucleic Acids Encoding Fusion Proteins and Delivery to Cells

Conventional viral and non-viral based gene transfer methods can be used to introduce nucleic acids encoding engineered TAL-cleavage domain fusion proteins in animal cells (e.g., mammalian cells) and target tissues. Such methods can also be used to administer nucleic acids encoding TAL-cleavage domain fusion proteins to cells in vitro. In certain embodiments, nucleic acids encoding TAL-cleavage domain fusion proteins are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1: 13-26 (1994).

Methods of non-viral delivery of nucleic acids encoding engineered TAL-cleavage domain fusion proteins include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.) and BTX Molecular Delivery Systems (Holliston, Mass.).

The use of RNA or DNA viral based systems for the delivery of nucleic acids encoding engineered TAL-cleavage domain fusion proteins take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of TAL-cleavage domain fusion proteins include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In applications in which transient expression of a TAL-cleavage domain fusion protein fusion protein is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and .psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a ZFP nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (see Inaba et al., J. Exp. Med. 176:1693-1702 (1992)).

Vectors (e.g., retroviruses, adenoviruses, liposomes, etc.) containing therapeutic TAL-cleavage domain fusion protein nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

With further respect to plants, the polynucleotides and vectors described herein can be used to transform a number of monocotyledonous and dicotyledonous plants and plant cell systems, including dicots such as safflower, alfalfa, soybean, coffee, amaranth, rapeseed (high erucic acid and canola), peanut or sunflower, as well as monocots such as oil palm, sugarcane, banana, sudangrass, com, wheat, rye, barley, oat, rice, millet, or sorghum. Also suitable are gymnosperms such as fir and pine.

Thus, the methods described herein can be utilized with dicotyledonous plants belonging, for example, to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. The methods described herein also can be utilized with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., Pinales, Ginkgoales, Cycadales and Gnetales.

The methods can be used over a broad range of plant species, including species from the dicot genera Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; the monocot genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, and Zea; or the gymnosperm genera Abies, Cunninghamia, Picea, Pinus, and Pseudotsuga.

A transformed cell, callus, tissue, or plant can be identified and isolated by selecting or screening the engineered cells for particular traits or activities, e.g., those encoded by marker genes or antibiotic resistance genes. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known. Polynucleotides that are stably incorporated into plant cells can be introduced into other plants using, for example, standard breeding techniques.

DNA constructs may be introduced into the genome of a desired plant host by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment (see, e.g., Klein et al (1987) Nature 327:70-73). Alternatively, the DNA constructs may be combined with suitable T-DNA flanking regions and introduced into a conventional Agrobacterium tumefaciens host vector. Agrobacterium tumefaciens-mediated transformation techniques, including disarming and use of binary vectors, are well described in the scientific literature. See, for example Horsch et al (1984) Science 233:496-498, and Fraley et al (1983) Proc. Nat'l. Acad. Sci. USA 80:4803. The virulence functions of the Agrobacterium tumefaciens host will direct the insertion of the construct and adjacent marker into the plant cell DNA when the cell is infected by the bacteria using binary T DNA vector (Bevan (1984) Nuc. Acid Res. 12:8711-8721) or the co-cultivation procedure (Horsch et al (1985) Science 227:1229-1231). Generally, the Agrobacterium transformation system is used to engineer dicotyledonous plants (Bevan et al (1982) Ann. Rev. Genet. 16:357-384; Rogers et al (1986) Methods Enzymol. 118:627-641). The Agrobacterium transformation system may also be used to transform, as well as transfer, DNA to monocotyledonous plants and plant cells. See Hernalsteen et al (1984) EMBO J. 3:3039-3041; Hooykass-Van Slogteren et al (1984) Nature 311:763-764; Grimsley et al (1987) Nature 325:1677-179; Boulton et al (1989) Plant Mol. Biol. 12:31-40; and Gould et al (1991) Plant Physiol. 95:426-434.

Alternative gene transfer and transformation methods include, but are not limited to, protoplast transformation through calcium-, polyethylene glycol (PEG)- or electroporation-mediated uptake of naked DNA (see Paszkowski et al. (1984) EMBO J3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618).

The disclosed methods and compositions can be used to insert exogenous sequences into a predetermined location in a plant cell genome. This is useful inasmuch as expression of an introduced transgene into a plant genome depends critically on its integration site. Accordingly, genes encoding, e.g., nutrients, antibiotics or therapeutic molecules can be inserted, by targeted recombination, into regions of a plant genome favorable to their expression.

Transformed plant cells which are produced by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al (1987) Ann. Rev. of Plant Phys. 38:467-486.

Nucleic acids introduced into a plant cell can be used to confer desired traits on essentially any plant. A wide variety of plants and plant cell systems may be engineered for the desired physiological and agronomic characteristics described herein using the nucleic acid constructs of the present disclosure and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Thus, the disclosed methods and compositions have use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea. One of skill in the art will recognize that after the expression cassette is stably incorporated in transgenic plants and confirmed to be operable, it can be introduced into other plants by sexual crossing. Any of a number of standard breeding techniques can be used, depending upon the species to be crossed.

A transformed plant cell, callus, tissue or plant may be identified and isolated by selecting or screening the engineered plant material for traits encoded by the marker genes present on the transforming DNA. For instance, selection may be performed by growing the engineered plant material on media containing an inhibitory amount of the antibiotic or herbicide to which the transforming gene construct confers resistance. Further, transformed plants and plant cells may also be identified by screening for the activities of any visible marker genes (e.g., the β-glucuronidase, luciferase, B or C1 genes) that may be present on the recombinant nucleic acid constructs. Such selection and screening methodologies are well known to those skilled in the art.

Physical and biochemical methods also may be used to identify plant or plant cell transformants containing inserted gene constructs. These methods include but are not limited to: 1) Southern analysis or PCR amplification for detecting and determining the structure of the recombinant DNA insert; 2) Northern blot, S1 RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

Effects of gene manipulation using the methods disclosed herein can be observed by, for example, northern blots of the RNA (e.g., mRNA) isolated from the tissues of interest. Typically, if the amount of mRNA has increased, it can be assumed that the corresponding endogenous gene is being expressed at a greater rate than before. Other methods of measuring gene and/or CYP74B activity can be used. Different types of enzymatic assays can be used, depending on the substrate used and the method of detecting the increase or decrease of a reaction product or by-product. In addition, the levels of and/or CYP74B protein expressed can be measured immunochemically, i.e., ELISA, RIA, EIA and other antibody based assays well known to those of skill in the art, such as by electrophoretic detection assays (either with staining or western blotting). The transgene may be selectively expressed in some tissues of the plant or at some developmental stages, or the transgene may be expressed in substantially all plant tissues, substantially along its entire life cycle. However, any combinatorial expression mode is also applicable.

The present disclosure also encompasses seeds of the transgenic plants described above wherein the seed has the transgene or gene construct. The present disclosure further encompasses the progeny, clones, cell lines or cells of the transgenic plants described above wherein said progeny, clone, cell line or cell has the transgene or gene construct.

### Delivery Vehicles

An important factor in the administration of polypeptide compounds, such as TAL-cleavage domain fusion protein, is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as TAL-cleavage domain fusion proteins across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996)). Another subsequence, the h (hydrophobic) domain of signal peptides, was found to have similar cell membrane translocation characteristics (see, e.g., Lin et al., J. Biol. Chem. 270:14255-14258 (1995)).

Examples of peptide sequences which can be linked to a protein, for facilitating uptake of the protein into cells, include, but are not limited to: an 11 amino acid peptide of the tat protein of HIV; a 20 residue peptide sequence which corresponds to amino acids 84-103 of the p16 protein (see Fahraeus et al., Current Biology 6:84 (1996)); the third helix of the 60-amino acid long homeodomain of Antennapedia (Derossi et al., J. Biol. Chem. 269:10444 (1994)); the h region of a signal peptide such as the Kaposi fibroblast growth factor (K-FGF) h region (Lin et al., supra); or the VP22 translocation domain from HSV (Elliot & O'Hare, Cell 88:223-233 (1997)). Other suitable chemical moieties that provide enhanced cellular uptake may also be chemically linked to ZFPs. Membrane translocation domains (i.e., internalization domains) can also be selected from libraries of randomized peptide sequences. See, for example, Yeh et al. (2003) Molecular Therapy 7(5):5461, Abstract #1191.

Toxin molecules also have the ability to transport polypeptides across cell membranes. Often, such molecules (called “binary toxins”) are composed of at least two parts: a translocation/binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used to deliver peptides to the cell cytosol as internal or amino-terminal fusions (Arora et al., J. Biol. Chem., 268:3334-3341 (1993); Perelle et al., Infect. Immun., 61:5147-5156 (1993); Stennark et al. J. Cell Biol. 113:1025-1032 (1991); Donnelly et al., PNAS 90:3530-3534 (1993); Carbonetti et al., Abstr. Annu. Meet. Am. Soc. Microbiol. 95:295 (1995); Sebo et al. Infect. Immun. 63:3851-3857 (1995); Klimpel et al. PNAS U.S.A. 89:10277-10281 (1992); and Novak et al., J. Biol. Chem. 267:17186-17193 1992)).

Such peptide sequences can be used to translocate TAL-cleavage domain fusion proteins across a cell membrane. TAL-cleavage domain fusion proteins can be conveniently fused to or derivatized with such sequences. Typically, the translocation sequence is provided as part of a fusion protein. Optionally, a linker can be used to link the TAL-cleavage domain fusion protein and the translocation sequence. Any suitable linker can be used, e.g., a peptide linker.

The TAL-cleavage domain fusion protein can also be introduced into an animal cell, preferably a mammalian cell, via a liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell,

The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, a TAL-cleavage domain fusion protein) at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane (see, e.g., PNAS 84:7851 (1987); Biochemistry 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

The disclosed methods for targeted recombination can be used to replace any genomic sequence with a homologous, non-identical sequence. For example, a mutant genomic sequence can be replaced by its wild-type counterpart, thereby providing methods for treatment of e.g., genetic disease, inherited disorders, cancer, and autoimmune disease. In like fashion, one allele of a gene can be replaced by a different allele using the methods of targeted recombination disclosed herein. Exemplary genetic diseases include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6.sup.th codon of beta-globin (HbC), hemophilia, Huntington's disease, Hurler Syndrome, hypophosphatasia, Kinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240).

Additional exemplary diseases that can be treated by targeted DNA cleavage and/or homologous recombination include acquired immunodeficiencies, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

In certain cases, alteration of a genomic sequence in a pluripotent cell (e.g., a hematopoietic stem cell) is desired. Methods for mobilization, enrichment and culture of hematopoietic stem cells are known in the art. See for example, U.S. Pat. Nos. 5,061,620; 5,681,559; 6,335,195; 6,645,489 and 6,667,064. Treated stem cells can be returned to a patient for treatment of various diseases including, but not limited to, SCID and sickle-cell anemia.

In many of these cases, a region of interest comprises a mutation, and the donor polynucleotide comprises the corresponding wild-type sequence. Similarly, a wild-type genomic sequence can be replaced by a mutant sequence, if such is desirable. For example, overexpression of an oncogene can be reversed either by mutating the gene or by replacing its control sequences with sequences that support a lower, non-pathologic level of expression. As another example, the wild-type allele of the ApoAl gene can be replaced by the ApoAl Milano allele, to treat atherosclerosis. Indeed, any pathology dependent upon a particular genomic sequence, in any fashion, can be corrected or alleviated using the methods and compositions disclosed herein.

Targeted cleavage and targeted recombination can also be used to alter non-coding sequences (e.g., regulatory sequences such as promoters, enhancers, initiators, terminators, splice sites) to alter the levels of expression of a gene product. Such methods can be used, for example, for therapeutic purposes, functional genomics and/or target validation studies.

### Example 1

### Chimeric Gene Construction

The chimeric gene for FN-AvrXa7 in a configuration of N-terminal FokI domain and C-terminal AvrXa7 was constructed using standard E. coli strains and DNA techniques (31). The full-length AvrXa7 was first modified with PCR primers Tal-F and Tal-R to integrate the restriction sites KpnI and BglII upstream of the start codon at 5′ end and HindIII, XbaI and a stop codon containing SpeI at 3′ end based on the plasmid pZWavrXa7 (29). AvrXa7 without repetitive central region was PCR amplified using primers Tal-F and Tal-R and cloned into pBluescript KS by KpnI and SpeI. Then the central repeat region was cloned back by SphI resulting in pSK/avrXa7. The DNA fragment encoding the cleavage domain (amino acids 388-583) of FokI (NCBI accession number J04623) was PCR amplified using the primers Fokn-F and Fokn-R and a plasmid containing FokI gene as template. Fokn-F contained the restriction sites KpnI and BglII, while Fokn-R contained a BamHI restriction sequence. The product was cloned into the A/T cloning vector pGEM-T (Promega, Madison). The KpnI and BamHI digested DNA fragment for FokI nuclease domain was cloned into KpnI and BglII treated pSK/avrXa7 resulting in pSK/FN-AvrXa7 which contained the chimeric gene with FN at 5′ and AvrXa7 at 3′ end. The accuracy of all PCR products was confirmed by sequencing. Primer sequences were provided in the Supplementary Data Table S1.

### Transient Expression Assay for DNA Binding Activity of FN-AvrXa7

The construct of reporter gene for green fluorescence protein (GFP) under the promoter of Os11N3 that contained the EBE of AvrXa7 was made as following. Region for GFP from plasmid pEGFP (Clontech Laboratories, Mountain View, Calif. 94043) was PCR amplified using primers GFP-F and GFP-R and cloned into pGEM-T for sequence confirmation. The GFP with added restriction sites was cloned between downstream of the promoter region containing the AvrXa7 EBE and upstream of the terminator of Os11N3 resulting in pEBE_{Os11N3}::GFP. The expression cassette of GFP was then cloned into pCAMBIA1300 (CAMBIA) at KpnI and HindIII restriction sites. The construct was transformed into Agrobacterium tumefaciens strain EHA105 as the reporter strain. DNA for FN-AvrXa7 was cloned under cauliflower mosaic virus (CaMV) 35S promoter in a modified pCAMBIA1300 vector and mobilized into EHA105 as effector strain. The effector strain containing AvrXa7 was similarly made as a positive control. The reporter and the effector strains were co-infiltrated into Nicotiana benthamiana leaves. The inoculated leaves were checked for expression of GFP under fluorescent stereomicroscope Leica M205 FA.

### Production and Purification of FN-AvrXa7

The chimeric gene FN-AvrXa7 was cloned into pPROEX HTb (Invitrogen) by ligating the BglII and SpeI digested FN-AvrXa7 fragment into BamHI and SpeI digested vector. The expression construct was transformed into E. coli strain BL21 (ED3) for overexpression of the recombinant protein with induction of isopropyl-1-thio-β-D-galactopyranoside (IPTG) following the manufacturer's manual (Invitrogen, Carlsbad, Calif. 92008). The 6Xhistidine tagged FN-AvrXa7 was purified with Ni-NTA agarose (Qiagen) and the protein concentration was determined using the BioRad's Bradford kit. The protein was loaded onto 10% SDS-polyacrylamide gels and performed protein gel blot analysis with a 1:20,000 dilution of anti-FLAG monoclonal antibody M2 (Sigma) to confirm the identity of AvrXa7 protein.

DNA Binding with Electromobility Shift Assay (EMSA)

The complementary oligonucleotides of Os11N3-F & Os11N3-R containing AvrXa7 EBE and Os11N3M-F & Os11N3M-R containing mutated AvrXa7 EBE were annealed, respectively, and were 5′-end labeled with [γ-³²P]ATP catalyzed by T4 polynucleotide kinase. The labeled oligonucleotide duplex DNA was mixed with FN-AvrXa7 in a reaction solution containing Tris-HCl (15 mM, pH 7.5), KCl (60 mM), DTT (1 mM), glycerol (2.0%), poly(dI.dC) (50 ng/ul), EDTA (0.2 mM), labeled DNA (50 fmol), FN-AvrXa7 (350 fmol) and, as competitor probes, unlabeled DNA (0-2.5 pmol). The binding reactions were kept at room temperature for 30 minutes before loaded on a 6% TBE polyacrylamide gel which was exposed to X-ray film for photograph after electrophoresis.

### In Vitro DNA Cleavage

A 406 bp genomic region of rice Os11N3 encompassing AvrXa7 EBE was PCR amplified with forward primer Os11N3P-F and Os11N3P-R and reverse primer and cloned into A/T cloning vector pTOPO (Invitrogen, Carlsbad, Calif. 92008). The clone was sequenced and linearized at the unique restriction site EcoNI that is located on the backbone of the plasmid before performing the in vitro digestion assay with FN-AvrXa7. The DNA (X ug) was incubated with FokI-AvrXa7 with the buffer condition same as for EMSA but in the presence of 2.5 mM MgCl₂.

### Yeast Recombination Assay

The yeast strains (YPH499 and YPH500) and expression plasmids (pCP5 and pCP3) were described and kindly provided by Dr. Dan Voytas (32). The pCP5 derived reporter construct containing a single AvrXa7 EBE was made by inserting the annealed oligonucleotides (EBES-F and EBES-R) into the BglII and SpeI digested pCP5. A duplex of oligonucleotides (EBEDH14-F and EBEDH14-R) containing two AvrXa7 EBEs in an orientation of head to head separated by a spacer of 14 bp was inserted into the BglII and SpeI digested pCP5. Similar constructs with two AvrXa7 EBEs but separated by various lengths (19, 24, 30, 35 bp) of spacers were made by swapping the first EBE with oligonucleotide duplexes of EBEDH19-F & EBEDH19-R, EBEDH24-F & EBEDH24-R, and, EBEDH30-F & EBEDH30-R, respectively, by BglII and KpnI. The replacements have the identical EBE sequences but different length of spacer nucleotides. The expression vector pCP3 was first modified with a linker sequence containing multiple cloning sites downstream of the translation elongation factor 1α promoter, resulting in pCP3M. The linker was made by annealing two oligonucleotides (Linker-F and Linker-R) and was ligated into the XbaI and XhoI digested pCP3. The chimeric gene FN-AvrXa7 was digested with BglII and SpeI and ligated into the BamHI and SpeI digested pCP3M vector. The reporter plasmids were transformed into the yeast mating strain YPH500 (MATα) and effector plasmids (FN-AvrXa7 and empty) into YPH499 (MATα). Transformants of the two mating strains were mixed and grown on yeast nutrient medium (YPD) overnight, then plated on synthetic complete medium lacking histidine and tryptophan. The colonies were membrane lifted and stained with X-gal containing Z buffer for β-galactosidase activity as described (33).

The primers and their sequences are provided in Supplemental data Table S1.

### Results

### Construction of the Chimeric Gene for FN-AvrXa7

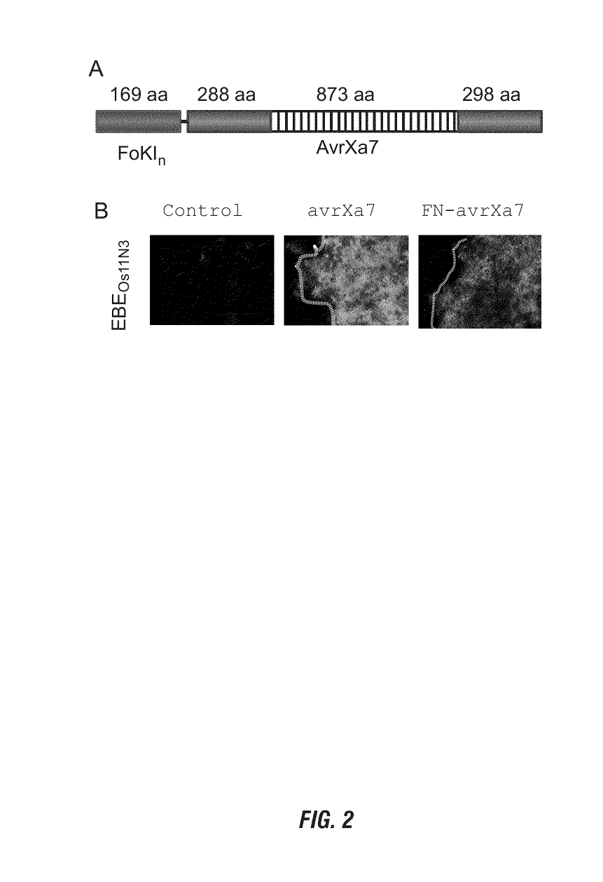

AvrXa7 is a naturally occurring TAL protein containing a central region of 26 repeat units but, like its relatives, the last repeat contains only first 20 amino acid residues similar to other repeats. The sequential sequence of its 26 RVDs makes itself a unique structure in comparison with other TAL proteins (FIG. 1B). AvrXa7 directly binds to a promoter element, specifically a predicted sequence of 26 base pairs in Os11N3 through its DNA binding repeats [FIG. 1 b; (34), also our submitted manuscript]. We reason that a hybrid protein of AvrXa7 and the DNA cleavage domain of an endonuclease may function in recognizing its target sequence and cleaving DNA adjacent to the recognition site. The DNA cleavage domain of the endonuclease FokI was chosen due to its well-documented nonspecific catalytic activity when linked with other DNA binding domains, such as zinc finger proteins. We chose a configuration of FN-AvrXa7 to make a chimeric gene by fusing the DNA sequence for the full-length AvrXa7 with the DNA sequence encoding the cleavage domain of FokI. The chimeric gene is predicted to encode a hybrid protein with FokI domain at N-terminus. The resulting chimeric gene is predicated to encode a protein of 1628 amino acid residues. The 196 amino acid FN is linked by 4 amino acid residues with AvrXa7 which by itself contains 1459 amino acids (FIG. 2A, also see Supplementary Figure S1 for the complete nucleotide and amino acid sequence of FN-AvrXa7).

### Transcription Activity of FN-AvrXa7 In Vivo

One reason we placed the FN at N-terminus and kept the TAL C-terminal activation domain intact in protein fusion was to investigate if we could take advantage of transcription activity as an indirect way to measure the DNA binding ability of the hybrid protein FN-AvrXa7, or any newly synthesized TAL derived hybrid protein in general. Since the full-length avrXa7 gene was used for the synthesis of chimeric gene, we expected that the hybrid protein still functioned as transcription activator when expressed in vivo. We adapted a modified Agrobacterium tumefaciens mediated in planta transient expression assay that was successfully used for studying interaction of TAL proteins with their target host genes (25, 28). In our case, the reporter construct contained the gene for green fluorescence protein (GFP) under the promoter of Os11N3 containing the AvrXa7 EBE; the effector constructs were made from AvrXa7 and FN-AvrXa7, respectively, under the strong and consecutive CaMV 35S promoter. Both reporter and effector genes were delivered by Agrobacterium tumefaciens and coexpressed in Nicotinana benthamiana leaves. Similarly to AvrXa7, the FN-AvrXa7 induced the expression of GFP while the construct lacking either avrXa7 or FN-avrXa7 did not (FIG. 2B). The results indicate that the hybrid FN-AvrXa7 retained the DNA binding ability of AvrXa7, and that the transient expression assay could provide a way to test the DNA binding ability of TAL-derived hybrid proteins in cells.

### Expression and Purification of FN-AvrXa7 Protein

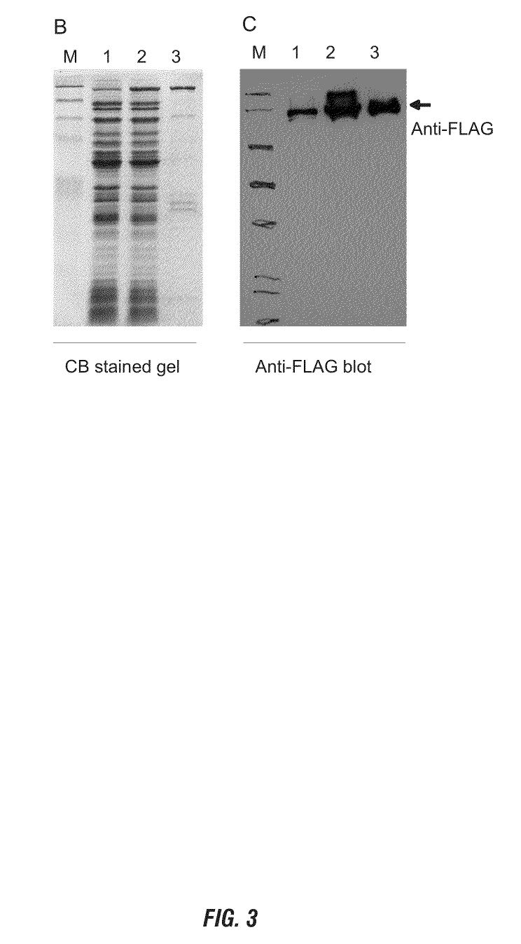

The chimeric gene FN-avrXa7 was cloned into overexpression vector in frame with a 6 histidine tag at the N-terminus for affinity chromatography purification from E. coli. The protein was successfully expressed from E. coli under induction of IPTG and purified with Ni beads for a relatively pure protein (FIG. 3A). The identity of FN-AvrXa7 was further confirmed by the western blot analysis using antibody against the FLAG epitode that was integrated into AvrXa7 at its C-terminus (Yang, et al 2000) (FIG. 3B). The expected size of protein is about 175 KD. With the addition of IPTG to the cultures, the E. coli cells expressing FN-AvrXa7 did not exhibit the obvious growth defect (Data not shown).

### DNA Binding and Cleavage Activity of FokI-AvrXa7

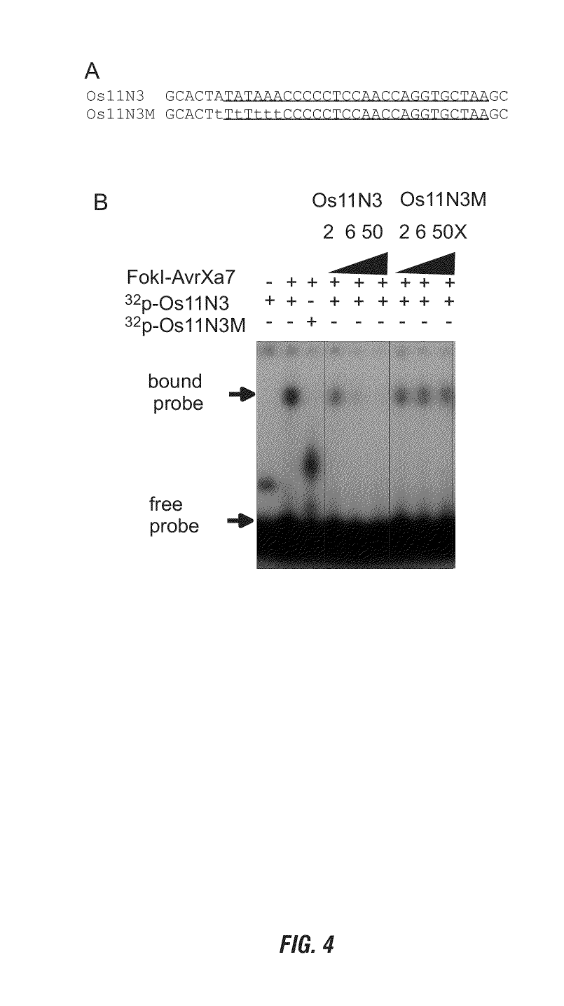

FN-AvrXa7 purified from E. coli was used to test its DNA binding specificity and catalytic activity in vitro. The ability of purified FN-AvrXa7 to bind DNA substrates in vitro was tested using oligonucleotide duplex containing the AvrXa7 EBE of Os11N3 and its mutated version (FIG. 4A). The electromobility shift assays (EMSA) demonstrated that FN-AvrXa7 preferentially binds to the labeled double stranded DNA containing target sequence but not to the probe containing the mutated target sequence (FIG. 4B, left panel with three lanes). Furthermore, the AvrXa7 EBE binding of FN-AvrXa7 could be competed with its unlabeled DNA probe, but binding was not competitive with excess of the variant oligonucleotide Os11N3M (FIG. 4B, middle and right panels).

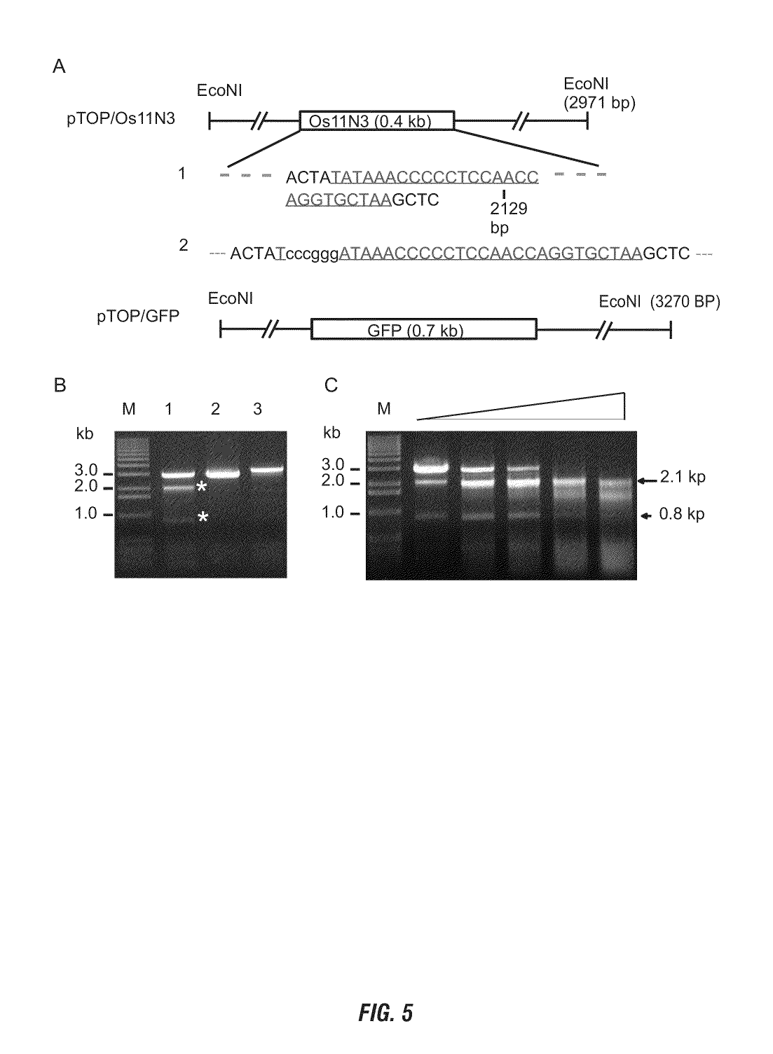

We also tested the ability of FN-AvrXa7 to cleave substrate DNA in vitro. We first chose a plasmid containing a cloned DNA fragment of Os11N3 promoter from rice. The plasmid pTOP/Os11N3 was first linearized at a unique restriction site (EcoN1) and purified after digestion. The plasmids containing the mutated AvrXa7 EBE site and an unrelated DNA fragment were used as control (FIG. 5A). The DNA was then incubated with FN-AvrXa7 at 37° C. for 1 hr under buffer condition as described. Clearly, the FN-AvrXa7 cleft the linearized DNA into two fragments indicative of one major cleavage site (FIG. 5B, lane 1), but not the plasmid containing a mutated binding site of AvrXa7 (FIG. 5B, lane 2), nor the plasmid containing GFP which is unrelated to the AvrXa7 target sequence (FIG. 5B, lane 3). The cleavage was also performed with increasing amount of FN-AvrXa7 protein. Under the concentration of X ng of FN-AvrXa7, the cleavage of X ng of substrate DNA could be complete, however, with increasing FN-AvrXa7, the cleavage appeared to be nonspecific as the smear bands showed up in the agarose gel (FIG. 5C). These experiments demonstrate that the FN-AvrXa7 has the enzymatic activity to cleave double stranded DNA and the cleavage activity is specific to the substrate sequence under certain reaction conditions.

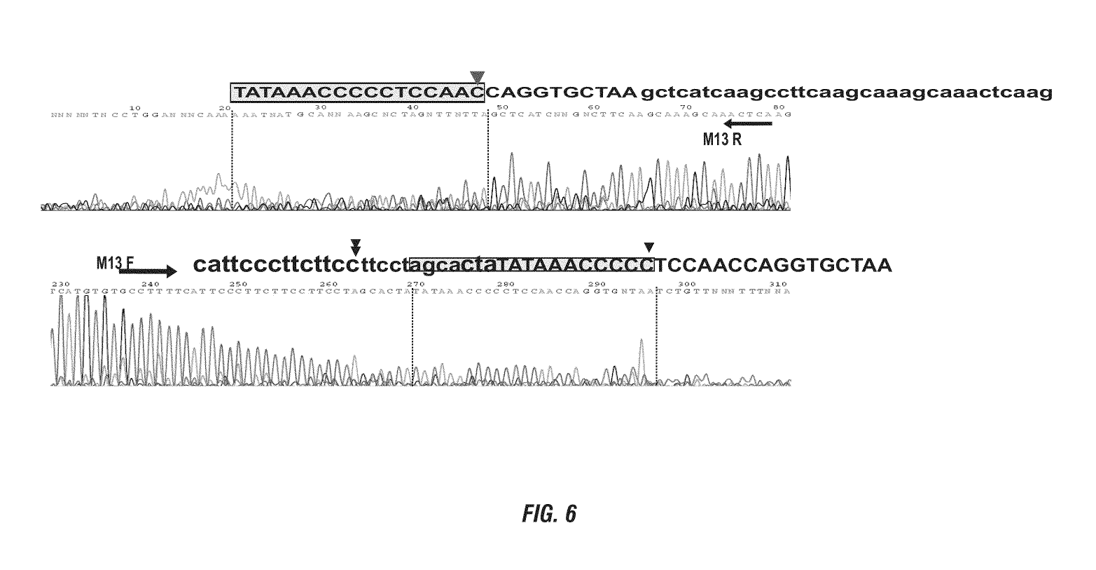

To identify the major cleavage sites of the sense and antisense strand, the cleaved DNA fragments (expected sizes of ˜890 bp and ˜2000 bp) derived from pTOP/Os11N3 were purified and subjected to sequencing by using two primers each complementary to one side of the 0.4 kb Os11N3 promoter fragment. The right side primer (M13R on pTOP) was used to sequence the sense strand which is the template of the prime. The reverse complementary sequence trace almost matched the original sequence of sense strand proximal to the AvrXa7 binding site whose trace poorly matched the original sequence (FIG. 6A). For the left side primer (M13F), the antisense strand was the template. The sequence trace perfectly matched the original sequence of sense strand including the binding sequence. Two major cleavage sites on the antisense strand could be interpreted from the chromatograph, one started at six base pairs upstream of binding site and another one located at the last nucleotide of binding site (FIG. 6B).

### FN-AvrXa7 Stimulated Homologous Recombination in Yeast

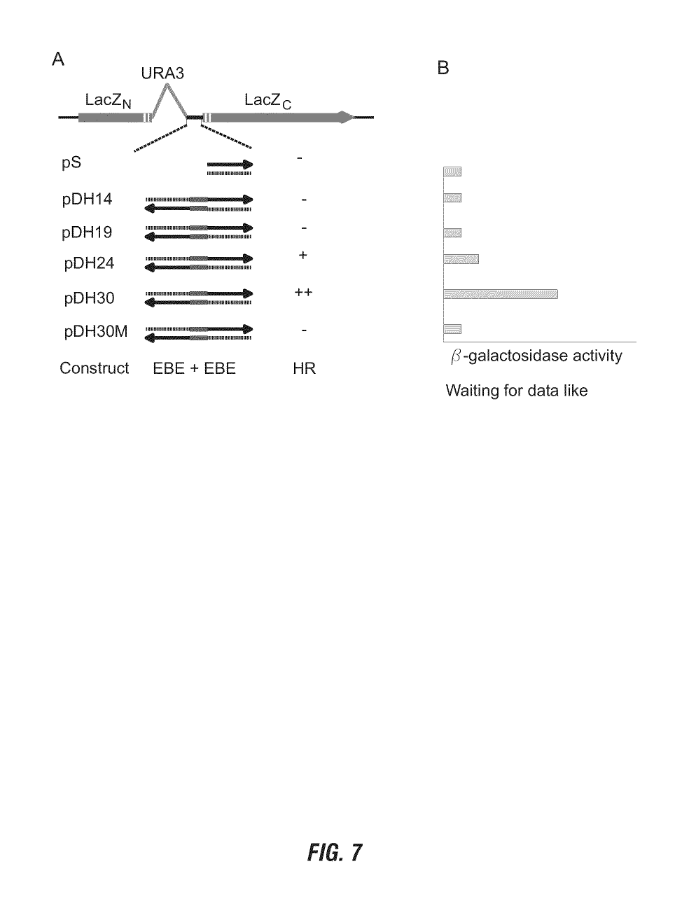

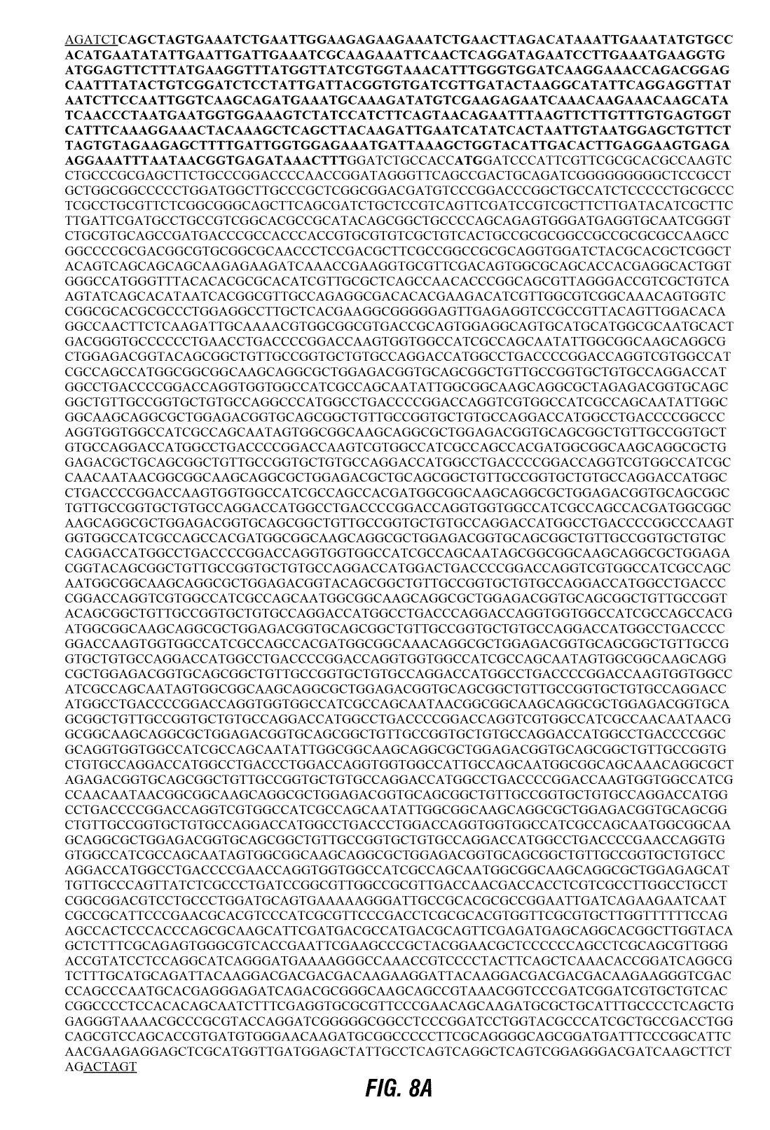

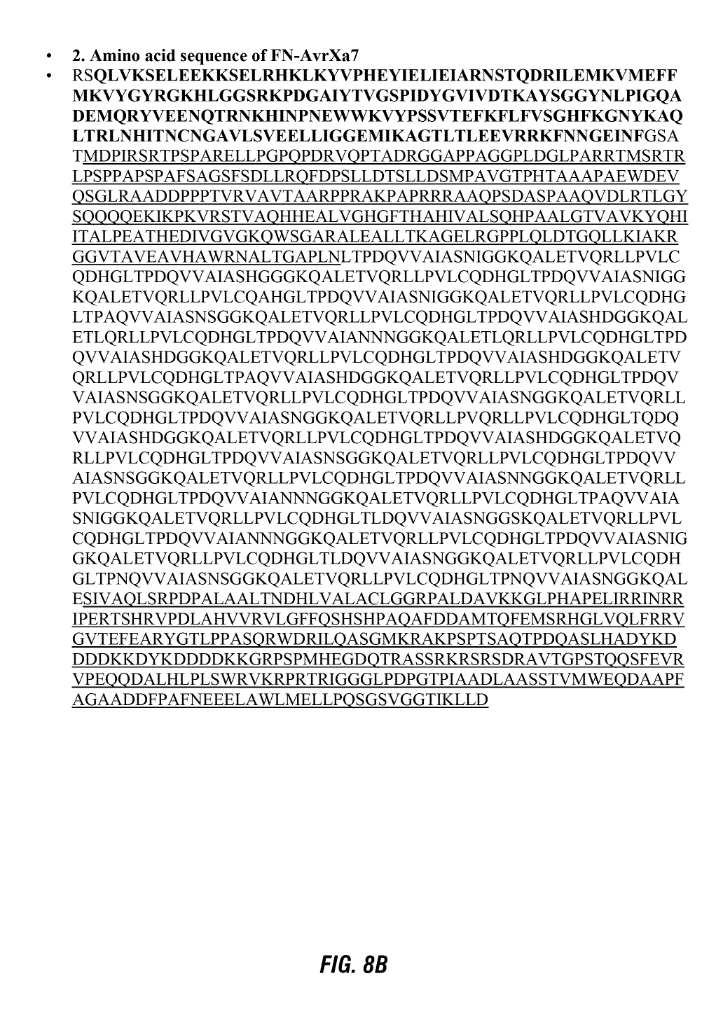

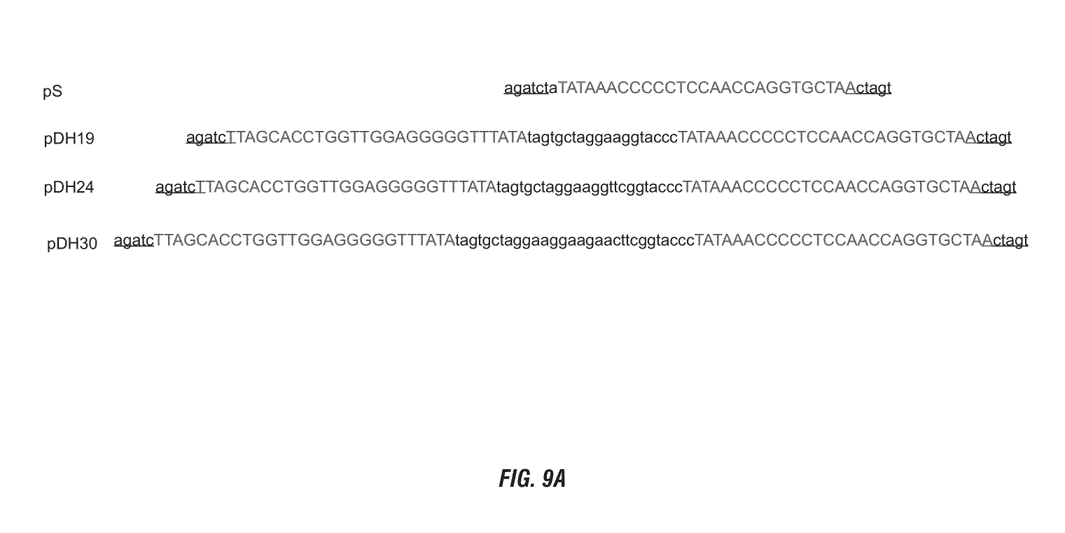

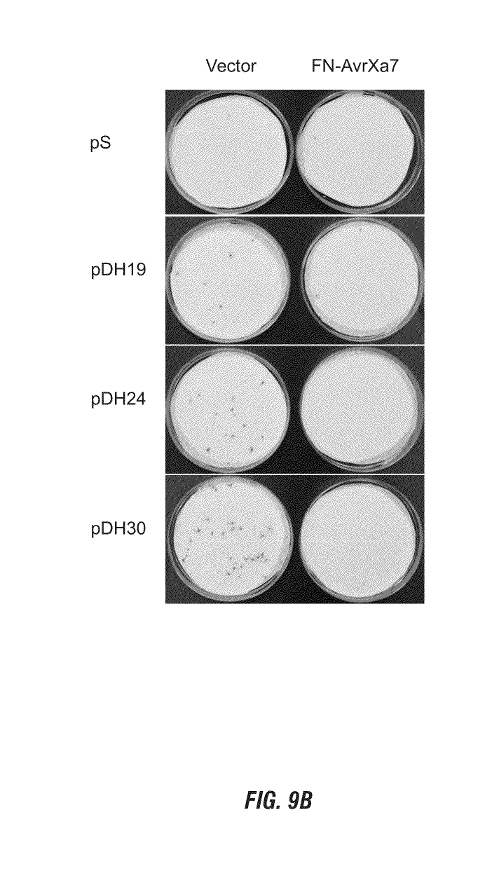

We sought to test the ability of the hybrid protein in binding and cleaving target sequence in vivo by using a previously established yeast single strand annealing assay (SSA) (32, 35). In this assay, a reporter construct is coexpressed with an effector construct in the yeast cells. The reporter construct contains two direct repeats of a 125 bp lacZ coding sequence that are separated by a 1.2 kb sequence encompassing the URA3 gene and a multiple cloning site for insertion of AvrXa7 EBE (FIG. 7A). The effector construct contains the FN-AvrXa7 under the TEF1 promoter. It is expected that the direct DNA repeats undergo homologous recombination in high efficiency when a cleavage between the repeats is generated, and the recombination results in the reconstitution of functional lacZ gene enabling the quantification of recombination frequency that reflects the functionality of TAL effector protein in the presence of target sequence (36, 37, 38).

A collection of reporter plasmids were constructed with one or two AvrXa7 EBE sites that were in an orientation of head-to-head and separated by variable lengths of spacers. Yeast cells with construct containing only one AvrXa7 EBE site did not show increased β-galactosidase activity when coexpressed with FN-AvrXa7 compared with the control that transformed with the effector construct lacking FN-AvrXa7 (FIG. 7 B, construct pS). Yeast cells transformed with plasmids containing 14 and 19 spacers between the two AvrXa7 EBE sites did not showed increased showed β-galactosidase activity either. However, the constructs containing 24 and 30 bp separated AvrXa7 EBE sites had significant amount of β-galactosidase activities than the control (FIG. 7B). The SSA assay demonstrates the FN-AvrXa7 efficiently cleaves the double strand DNA at the paired sites in yeast cells.

### DISCUSSION

Years' efforts trying to understand the interaction between TAL effectors and their modulated host genes have led to the recent breakthrough in deciphering the DNA recognition code of TAL effectors (27, 28). The predictability and manipulability of TAL central domain for DNA binding specificities make TAL an excellent system for exploiting potential biotechnological applications. In present study we tested the amenability of TAL DNA binding activity in fusion with functional domain of other proteins. AvrXa7, a typical TAL with known target sequence specificity, was chosen to create a chimeric protein by linking it to C-terminus of the FokI nuclease domain. The recombinant protein has been successfully produced and purified from E. coli cells and exhibited cleavage activity at expected site in the optimized reaction conditions. The hybrid protein when expressed stimulated the HR of a reporter gene (LacZ) that contained the paired recognition sites in a yeast single stranded annealing assay.

FokI has been extensively studied (13, 14, 15). The endonuclease domain by itself has no specificity for cleavage, but incises DNA at a site specified by the DNA binding domain when linked together. In this sense, several types of Fokln based fusion proteins have been successfully created that retain new sequence specificities and cleavage activities with the ZFNs the most popular (6, 7, 39). We chose the FokI cleavage domain to fuse with one member of TAL effector family and, as a proof of principle, demonstrated the feasibility of creating a kind of nucleases with sequence specificities that can be attributable to the TAL effectors. The features of TAL effectors for DNA binding make this group of proteins or their repetitive domains desirable as the key component of endonucleases when fused with nonspecific DNA cleavage domains for some applications including genome editing. For example, the majority of naturally occurring TAL proteins contains a large number of repeat units and correspondingly recognizes, as demonstrated in few cases, longer sequences that are comparable to the lengths of target sties of rare-cutting meganucleases or homing nucleases (14 to 40 bp) as well as artificial ZFPs assembled from multiple single fingers (−18 bp) (5, 40). The TAL proteins that have ever been investigated exhibit high sequence specificity to the EBEs of their target genes (34, 41). Furthermore, the model for predicting target sites of TAL protein based on the numbers and RVD characters of repeat units may be reversely used to design TAL proteins based on the DNA sequence of interest, a modular feature amenable to manipulation. The next step is to test the feasibility of custom-engineering novel TAL proteins capable of recognizing a large range of DNA sites with high specificity and affinity.

So far, TAL effectors have been found to function as transcription activators and, like many other transcription factors, act probably as dimers to bind target DNA. AvrBs3 is the only one TAL effector that was indicated to dimerize in vitro and in cytoplasm before entry into nuclei of host cells (43). The sequence specificity of known TAL can be aligned to only one strand of the target site and the sequence generally is asymmetric (27, 28). It is not clear if TAL effector proteins in general form dimers or multimers in the presence of target DNA or lack thereof. The structure studies on TAL effectors will help answer such questions as if the intermolecular reaction exists. However, AvrXa7-Fokln could recognize single

Distance between two recognition sites seems flexible as for ZFNs tested which is in a range of >4 and <40 (44). It has been established that for efficient double strand cleavage of target DNA, the Fokln dimerization is required (17). Therefore, it is conceivable Fokln-AvrXa7 needs to dimerize for the efficient incision of DNA. This could be achieved through two models presented below. First, one EBE-bound Fokln-AvrXa7 forms a dimer with another free or the readily bound Fokln-AvrXa7 through an as yet uncharacterized dimerization domain of TAL effectors, and the AvrXa7-mediated dimerization brings the two FokI nuclease domains in close vicinity at the binding site for cleavage under our in vitro cleavage condition. Alternatively, similarly to those for ZFNs and native FokI, the two EBE bound FokI-AvrXa7 form dimer through the Fokln for an effective double strand cleavage. The native FokI function is allosterically regulated through DNA and divalent metal binding. It is possible that the hybrid nuclease lacks such regulation and is more relaxed in executing the cleavage function of FokI nuclease domain. Without binding and in the absence of divalent metal, Fokln is sequestered through interaction with the DNA recognition motifs and, thus, FokI monomer maintains an idle state. Following binding to the recognition site and in the presence of metals, two readily bound FokI individual molecules form a dimer through the interaction of the cleavage domains. The dimerization brings the two DNA/protein complexes in close proximity for a double strand incision (14, 16, 17). The linked Fokln does not alter the sequence specificity of DNA binding partner as in the case of ZFNs (5). Fokln-AvrXa7 showed multiple cleaving sites on both strands around the AvrXa7 EBE site. It is possible that region between the repeat region for binding function and Fokln which is about 300 amino acid residues makes the domain relaxed for cutting. Similar findings of multiple cuts were also observed for ZFNs and even native type IIS enzymes (7, 12, 44).

### REFERENCES

1. Le Provost, F., Lillico, S., Passet, B., Young, R., Whitelaw, B. and Vilotte, J. L. (2010) Zinc finger nuclease technology heralds a new era in mammalian transgenesis. Trends Biotechnol., 28, 134-141.
2. Jasin, M. (1996) Genetic manipulation of genomes with rare-cutting endonucleases. Trends Genet., 12, 224-228.
3. Vasquez, K. M., Marburger, K., Intody, Z. and Wilson, J. H. (2001) Manipulating the mammalian genome by homologous recombination. Proc. Natl. Acad. Sci. U.S. A., 98, 8403-8410.
4. Bibikova, M., Beumer, K., Trautman, J. K. and Carroll, D. (2003) Enhancing gene targeting with designed zinc finger nucleases. Science, 300, 764.
5. Porteus, M. H. and Carroll, D. (2005) Gene targeting using zinc finger nucleases. Nat. Biotechnol., 23, 967-973.
6. Kim, Y. G. and Chandrasegaran, S. (1994) Chimeric restriction endonuclease. Proc. Natl. Acad. Sci. U.S.A., 91, 883-887.
7. Kim, Y. G., Cha, J. and Chandrasegaran, S. (1996) Hybrid restriction enzymes: Zinc finger fusions to fok I cleavage domain. Proc. Natl. Acad. Sci. U.S.A., 93, 1156-1160.
8. Isalan, M. and Choo, Y. (2001) Engineering nucleic acid-binding proteins by phage display. Methods Mol. Biol., 148, 417-429.
9. Pabo, C. O., Peisach, E. and Grant, R. A. (2001) Design and selection of novel Cys2H is2 zinc finger proteins. Annu. Rev. Biochem., 70, 313-340.
10. Beerli, R. R. and Barbas, C. F., 3rd. (2002) Engineering polydactyl zinc-finger transcription factors. Nat. Biotechnol., 20, 135-141.
11. Sugisaki, H. and Kanazawa, S. (1981) New restriction endonucleases from flavobacterium okeanokoites (FokI) and micrococcus luteus (MluI). Gene, 16, 73-78.
12. Szybalski, W., Kim, S. C., Hasan, N. and Podhajska, A. J. (1991) Class-IIS restriction enzymes—a review. Gene, 100, 13-26.
13. Li, L., Wu, L. P. and Chandrasegaran, S. (1992) Functional domains in fok I restriction endonuclease. Proc. Natl. Acad. Sci. U.S.A., 89, 4275-4279.
14. Vanamee, E. S., Santagata, S. and Aggarwal, A. K. (2001) FokI requires two specific DNA sites for cleavage. J. Mol. Biol., 309, 69-78.
15. Wah, D. A., Hirsch, J. A., Dorner, L. F., Schildkraut, I. and Aggarwal, A. K. (1997) Structure of the multimodular endonuclease FokI bound to DNA. Nature, 388, 97-100.
16. Wah, D. A., Bitinaite, J., Schildkraut, I. and Aggarwal, A. K. (1998) Structure of FokI has implications for DNA cleavage. Proc. Natl. Acad. Sci. U.S.A., 95, 10564-10569.
17. Bitinaite, J., Wah, D. A., Aggarwal, A. K. and Schildkraut, I. (1998) FokI dimerization is required for DNA cleavage. Proc. Natl. Acad. Sci. U.S.A., 95, 10570-10575.
18. Cathomen, T. and Joung, J. K. (2008) Zinc-finger nucleases: The next generation emerges. Mol. Ther., 16, 1200-1207.
19. Ramirez, C. L., Foley, J. E., Wright, D. A., Muller-Lerch, F., Rahman, S. H., Cornu, T. I., Winfrey, R. J., Sander, J. D., Fu, F., Townsend, J. A., et al. (2008) Unexpected failure rates for modular assembly of engineered zinc fingers. Nat. Methods, 5, 374-375.
20. Kim, J. S., Lee, H. J. and Carroll, D. (2010) Genome editing with modularly assembled zinc-finger nucleases. Nat. Methods, 7, 91; author reply 91-2.
21. White, F. F., Potnis, N., Jones, J. B. and Koebnik, R. (2009) The type III effectors of xanthomonas. Mol. Plant. Pathol., 10, 749-766.
22. Gu, K., Yang, B., Tian, D., Wu, L., Wang, D., Sreekala, C., Yang, F., Chu, Z., Wang, G. L., White, F. F., et al. (2005) R gene expression induced by a type-III effector triggers disease resistance in rice. Nature, 435, 1122-1125.
23. Yang, B., Sugio, A. and White, F. F. (2006) Os8N3 is a host disease-susceptibility gene for bacterial blight of rice. Proc. Natl. Acad. Sci. U.S.A., 103, 10503-10508.
24. Sugio, A., Yang, B., Zhu, T. and White, F. F. (2007) Two type III effector genes of Xanthomonas oryzae pv. oryzae control the induction of the host genes OsTFIIAgamma1 and OsTFX1 during bacterial blight of rice. Proc. Natl. Acad. Sci. U.S.A., 104, 10720-10725.
25. Romer, P., Hahn, S., Jordan, T., Strauss, T., Bonas, U. and Lahaye, T. (2007) Plant pathogen recognition mediated by promoter activation of the pepper Bs3 resistance gene. Science, 318, 645-648.
26. Gurlebeck, D., Thieme, F. and Bonas, U. (2006) Type III effector proteins from the plant pathogen xanthomonas and their role in the interaction with the host plant. J. Plant Physiol., 163, 233-255.
27. Moscou, M. J. and Bogdanove, A. J. (2009) A simple cipher governs DNA recognition by TAL effectors. Science, 326, 1501.
28. Boch, J., Scholze, H., Schornack, S., Landgraf, A., Hahn, S., Kay, S., Lahaye, T., Nickstadt, A. and Bonas, U. (2009) Breaking the code of DNA binding specificity of TAL-type III effectors. Science, 326, 1509-1512.
29. Yang, B., Zhu, W., Johnson, L. B. and White, F. F. (2000) The virulence factor AvrXa7 of xanthomonas oryzae pv. oryzae is a type III secretion pathway-dependent nuclear-localized double-stranded DNA-binding protein. Proc. Natl. Acad. Sci. U.S.A., 97, 9807-9812.
30. Hopkins, C. M., White, F. F., Choi, S. H., Guo, A. and Leach, J. E. (1992) Identification of a family of avirulence genes from xanthomonas oryzae pv. oryzae. Mol. Plant. Microbe Interact., 5, 451-459.
31. Sambrook, J., Fritsch, E. F. and Maniatis, T. (1987) Molecular Cloning: A Laboratory Manual. Cold Pring Harbor Laboratory Press, Cold Spring Harbor, N.Y., U.S.A.
32. Townsend, J. A., Wright, D. A., Winfrey, R. J., Fu, F., Maeder, M. L., Joung, J. K. and Voytas, D. F. (2009) High-frequency modification of plant genes using engineered zinc-finger nucleases. Nature, 459, 442-445.
33. Wright, D. A., Thibodeau-Beganny, S., Sander, J. D., Winfrey, R. J., Hirsh, A. S., Eichtinger, M., Fu, F., Porteus, M. H., Dobbs, D., Voytas, D. F., et al. (2006) Standardized reagents and protocols for engineering zinc finger nucleases by modular assembly. Nat. Protoc., 1, 1637-1652.
34. Romer, P., Recht, S., Strauss, T., Elsaesser, J., Schornack, S., Boch, J., Wang, S. and Lahaye, T. (2010) Promoter elements of rice susceptibility genes are bound and activated by specific TAL effectors from the bacterial blight pathogen, Xanthomonas oryzae pv. oryzae. New Phytol., 10.1111/j.1469-8137.2010.03217.x.
35. Epinat, J. C., Arnould, S., Chames, P., Rochaix, P., Desfontaines, D., Puzin, C., Patin, A., Zanghellini, A., Paques, F. and Lacroix, E. (2003) A novel engineered meganuclease induces homologous recombination in yeast and mammalian cells. Nucleic Acids Res., 31, 2952-2962.
36. Rudin, N. and Haber, J. E. (1988) Efficient repair of HO-induced chromosomal breaks in saccharomyces cerevisiae by recombination between flanking homologous sequences. Mol. Cell. Biol., 8, 3918-3928.
37. Sugawara, N. and Haber, J. E. (1992) Characterization of double-strand break-induced recombination: Homology requirements and single-stranded DNA formation. Mol. Cell. Biol., 12, 563-575.
38. Fishman-Lobell, J., Rudin, N. and Haber, J. E. (1992) Two alternative pathways of double-strand break repair that are kinetically separable and independently modulated. Mol. Cell. Biol., 12, 1292-1303.
39. Kim, Y. G., Smith, J., Durgesha, M. and Chandrasegaran, S. (1998) Chimeric restriction enzyme: Ga14 fusion to FokI cleavage domain. Biol. Chem., 379, 489-495.
40. Belfort, M. and Roberts, R. J. (1997) Homing endonucleases: Keeping the house in order. Nucleic Acids Res., 25, 3379-3388.
41. Romer, P., Recht, S. and Lahaye, T. (2009) A single plant resistance gene promoter engineered to recognize multiple TAL effectors from disparate pathogens. Proc. Natl. Acad. Sci. U.S.A., 106, 20526-20531.
42. Gurlebeck, D., Szurek, B. and Bonas, U. (2005) Dimerization of the bacterial effector protein AvrBs3 in the plant cell cytoplasm prior to nuclear import. Plant J., 42, 175-187.
43. Smith, J., Bibikova, M., Whitby, F. G., Reddy, A. R., Chandrasegaran, S. and Carroll, D. (2000) Requirements for double-strand cleavage by chimeric restriction enzymes with zinc finger DNA-recognition domains. Nucleic Acids Res., 28, 3361-3369.

TABLE S1
Primers used in this study

| | |
|---|---|
| Fokn-F | 5′-CCATGGTACCAGATCTCAGCTAGTGAAATCTGAATT |
| | GG-3′ |
| Fokn-R | 5′-CCGGATCCAAAGTTTATCTCACCGTTATTAAATTT |
| | C-3′ |
| GFP-F | 5′-CACCAGATCTCGCCACCATGGTGAGCAAGGG-3′ |
| GFP-R | 5′-CCGGATCCTCCGGACTTGTACAGCTCGTC-3′ |
| TAL-F | 5′-CACCGGTACCAGATCTGCCACCATGGATCCCATTCG |
| | TTCGCGCAC-3′ |
| TAL-R | 5′-CCACTAGTCTAGAAGCTTGATCGTCCCTCCGACTGA |
| | GCCTG-3′ |
| Os11N3-F | 5′-GCACTATATAAACCCCCTCCAACCAGGTGCTAAG |
| | C-3′ |
| Os11N3-R | 5′-GCTTAGCACCTGGTTGGAGGGGGTTTATATAGTG |
| | C-3′ |
| Os11N3M-F | 5′-GCACTTTTTTTTCCCCCTCCAACCAGGTGCTAAG |
| | C-3′ |
| Os11N3M-R | 5′-GCTTAGCACCTGGTTGGAGGGGGAAAAAAAAGTG |
| | C-3′ |
| Os11N3P-F | 5′-CACCGGTACCATGGCTGTGATTGATCAGG-3′ |
| Os11N3P-R | 5′-CCGGATCCAGCCATTGCAGCAAGATCTTG-3′ |
| EBES-F | 5′-GATCTATATAAACCCCCTCCAACCAGGTGCTAA-3′ |
| EBES-R | 5′-CTAGTTAGCACCTGGTTGGAGGGGGTTTATATA-3′ |
| EBEDH19-F | 5′-GATCTTAGCACCTGGTTGGAGGGGGTTTATATAGTG |
| | CTAGGAAGGTAC-3′ |
| EBEDH19-R | 5′-CTTCCTAGCACTATATAAACCCCCTCCAACCAGGTG |
| | CTAA-3′ |
| EBEDH24-F | 5′-GATCTTAGCACCTGGTTGGAGGGGGTTTATATAGTG |
| | CTAGGAAGGTTCGGTAC-3′ |
| EBEDH24-R | 5′-CGAACCTTCCTAGCACTATATAAACCCCCTCCAACC |
| | AGGTGCTAA-3′ |
| EBEDH30-F | 5′-GATCTGTGGTGTACAGTAGGGGGAGATGCATATCTA |
| | ACCTTTGCTTTTTTTTCGGTAC-3′ |
| EBEDH30-R | 5′-CGAAAAAAAAGCAAAGGTTAGATATGCATCTCCCCC |
| | TACTGTACACCAC-3′ |
| Linker-F | 5′-CTAGAGGATCCGTCGACAAGCTTACTAGTC-3′ |
| Linker-R | 5′-TCGAGACTAGTAAGCTTGTCGACGGATCCT-3′ |

### Example 2

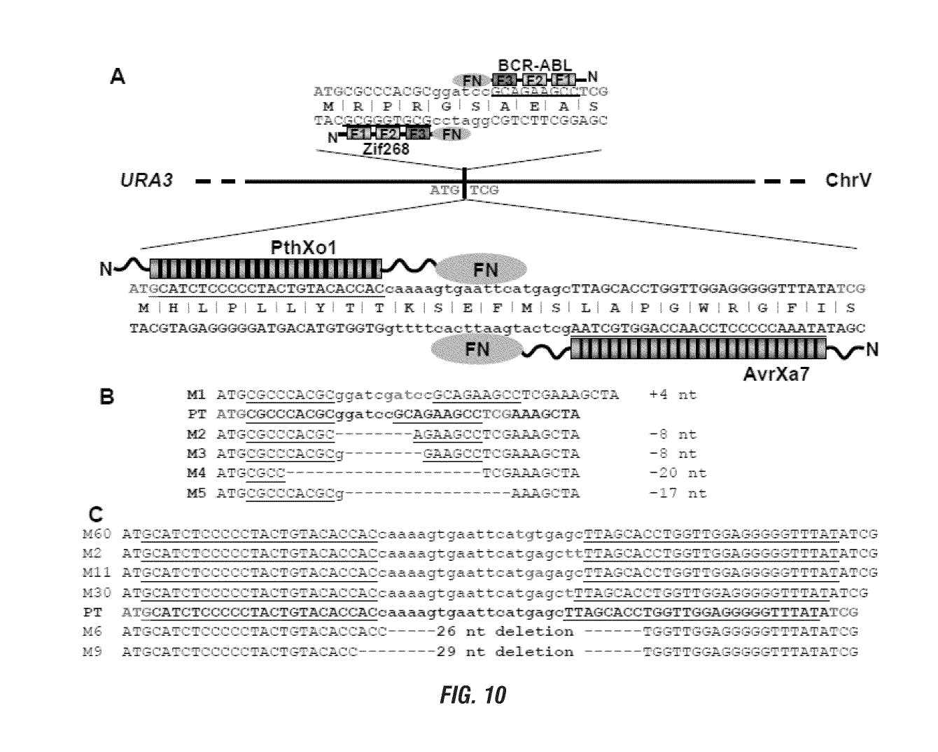

TALNs derived from the native TAL effectors target their EBEs in yeast chromosomal context. More recently, our results have demonstrated the feasibility of gene disruptions caused by TALNs when targeted to genes on yeast chromosome (vs. yeast plasmid DNA demonstrated previously) by constructing a URA3 gene containing PthXol/AvrXa7 EBE sites immediately downstream of the gene's ATG start codon and replacing the wild type URA3 gene on chromosome 5 with this modified, but fully functional, URA3 gene. Similarly, the dual target sequence for a pair of known ZFNs was also integrated into the URA3 gene for comparison (FIG. 1A). Yeast cells in which the URA3 gene was inactivated were selected on media containing 5-fluoroorotic acid (5-FOA), which is converted to a toxin in cells containing a functional URA3 gene. Results shown in FIGS. 10B & 10C demonstrated that expression of both types of nucleases in transformed yeast cells resulted in specific cleavage at the targeted sites and mutagenic DNA insertions/deletions due to the error-prone NHEJ to the DSBs.

FIG. 10. (A) Schematics of yeast URA3 gene in chromosome 5 (ChrV) with the integrated targeted sequences in frame with the ORF of URA3 gene. The target sites are underlined with the spacer sequence in lower case letters. The ZFNs and TALNs bind to the target sites and the FokI nuclease domains (FN) dimerize and cleave double stranded DNA between the target sites. (B) Genomic DNA sequences at the sites of mutations induced by ZFNs. Parental strain (PT) and five representatives of mutants (M) with insertion (red lower case letter) and deletions (red dashes) were shown. (C) Genomic sequences at the sites of mutations caused by TALNs. The lower case letters in red indicate insertions and the dashed lines denote DNA sequences deleted in the mutants (M) compared to the parental strain (PT).

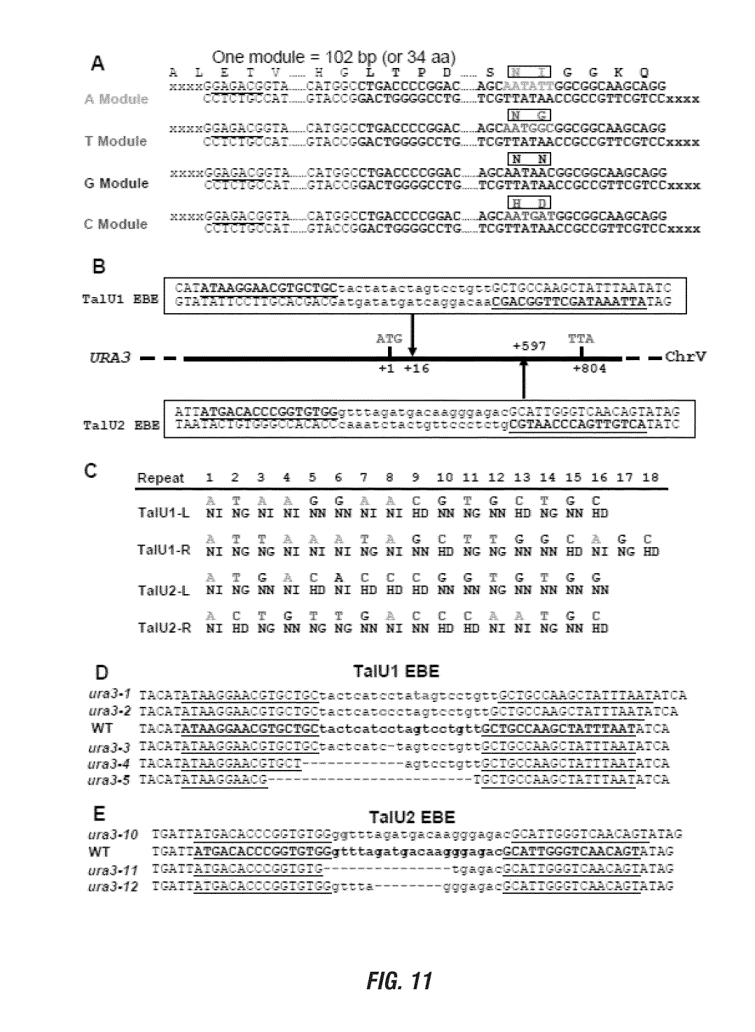

Amenability of custom-engineering TALNs by assembling four modules and ability of artificial TALNs in making targeted DSBs and subsequent genetic modification to the endogenous genes in yeast. Other recent, unpublished, experiments have allowed us to demonstrate that genes in their native chromosomal context can be successfully targeted for knockout using artificial TALNs whose central 34 AA repeat units are encoded by genes synthesized and assembled in vitro. FIG. 11A shows the four modules encoding 34 AA repeat units designed to recognize each of the four nucleotides in DNA (A, T, G and C). PCR amplification of these modules using primers designed to produce unique 4 base pair overhangs at each end followed with digestion of the restriction enzyme, BsmBI, results in a collection of “repeat modules” that can be uniquely assembled into a gene that encodes a TAL effector capable of recognizing a specific DNA sequence. In the experiments depicted in FIG. 11, two sites in the wild type URA3 gene (at positions +16 and +597) were selected for separate targeting by two different sets of TALNs designed to recognize the respective targeted sequences (FIG. 11C). Transformation of wild type yeast cells with plasmids containing either set of TALN genes resulted in the production of colonies able to grow in the presence of 5-FOA. Cells transformed with sets of plasmids lacking the TALN genes produced no 5-FOA resistant colonies (data not shown). DNA sequencing analyses revealed a variety of deletions/insertions caused by the two sets of TALNs (representative data are provided in FIG. 11D, 11E).

FIG. 11. (A) Four modules each encoding 34 AA with the twelfth and thirteenth residues (RVD) that specifically recognize one of the four nucleotides (i.e., NI for A, NG for T, NN for G, and HD for C, respectively). Each module consists of two halves of adjacent repeats (2nd half in bold). The 4 base pair overhangs (XXXX) at each end are generated by BsmBI whose recognition site is GAGACG (underlined). The 4 bp overhangs are compatible with the overhangs of adjacent repeat units on either side—thus allowing sequential assembly of the 102 bp repeats and the resulting TAL effector match an array of specific nucleotides in the target gene. Dots denote nucleotides or amino acids not shown. (B) Two EBE sites at positions +16 and +597 (relative to the “A” of the ATG start codon) of the yeast URA3 gene (region delimited by red typeface ATG and TTA) on chromosome 5 (ChrV) chosen as target sites (boxed sequences underlined) for engineering TALNs (TalU1-L and TalU1-R for the EBE site beginning at +16 and TalU2-L and TalU2-R for the position at +597). (C) The RVD sequences of the four TALNs (TalU1-L, —R, and TalU2-L, —R) and their corresponding recognition DNA sequences are shown with the sequential order of repeats that were custom-synthesized using the individual modules illustrated in (A). (D) and (E) DNA alignment of URA3 alleles retrieved from the parental strain (WT) and its derivative mutants (ura3-1, -2, -3, -4, -5, -10, -11 and -12) with insertions (red letters)/deletions (dashes in red) relevant to two sets of TALNs (TalU1-L, -R and TalU2-L, -R). The dual TALN target sites (TalU1 EBE and TalU2 EBE) are underlined.

### Example 3

### Targeted Gene Disruption in Mammalian Cells

Applicants designed a TALE endonuclease that targets the reporter gene Green Fluorescent Protein (egfp). This was accomplished by cloning eGFP dTALENs into a mammalian expression vector and transfecting human HEK293T cells with EGFP expression plasmid in the presence of increasing amounts of eGFP dTALENS. Next the GFP transfected cells were quantified by FACS. Then the GFP gene was amplified and sequenced from treated cells to characterize mutations/insertions at the target site.

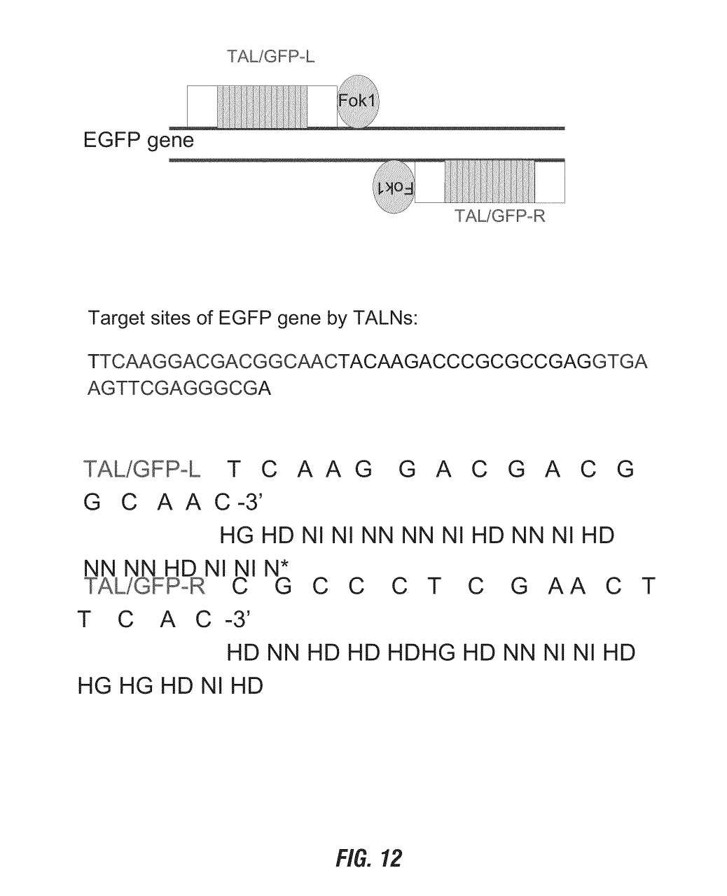

FIG. 12 shows the target sites of the eGFP gene by TALNs.

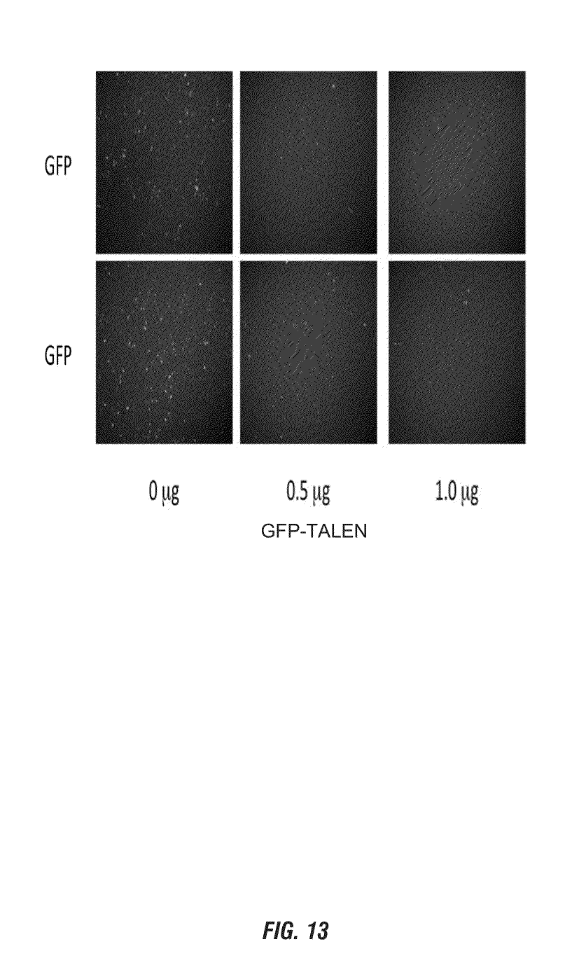

For transfection of the human HEK293T cells with EGFP expression plasmid in the presence of increasing amounts of eGFP dTALEN, the HEK293-T cells were plated in 6-well plate. The cells were co-transfected with the DNAs at pEGFP-c2: 100 ng/well and TAL/GFP-L+TAL/GFP-R at 0, 0.5, lug/well (in duplicate). The cells were then incubated for 3 days, and examined with fluorescent microscope. FIG. 13 shows the GFP detection.

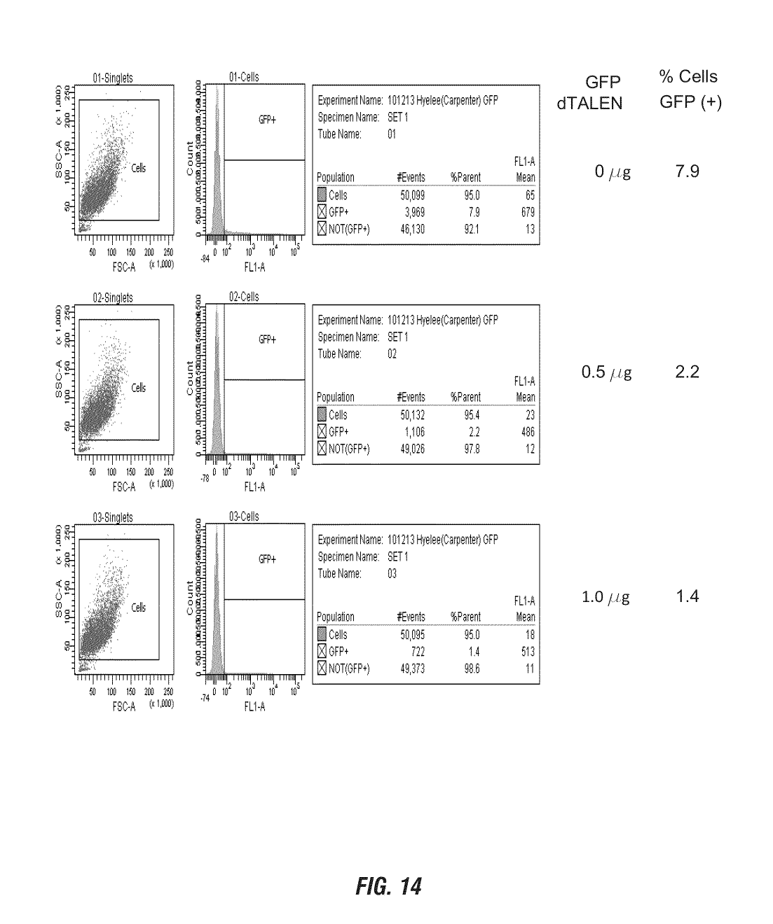

Next to quantify GFP-transfected cells by FACS, the cells were detached from E-well plate and fixed in paraformaldehyde. 50,000 cells from each treatment group were analyzed by FACS for GFP expression. The results are shown in FIG. 14.

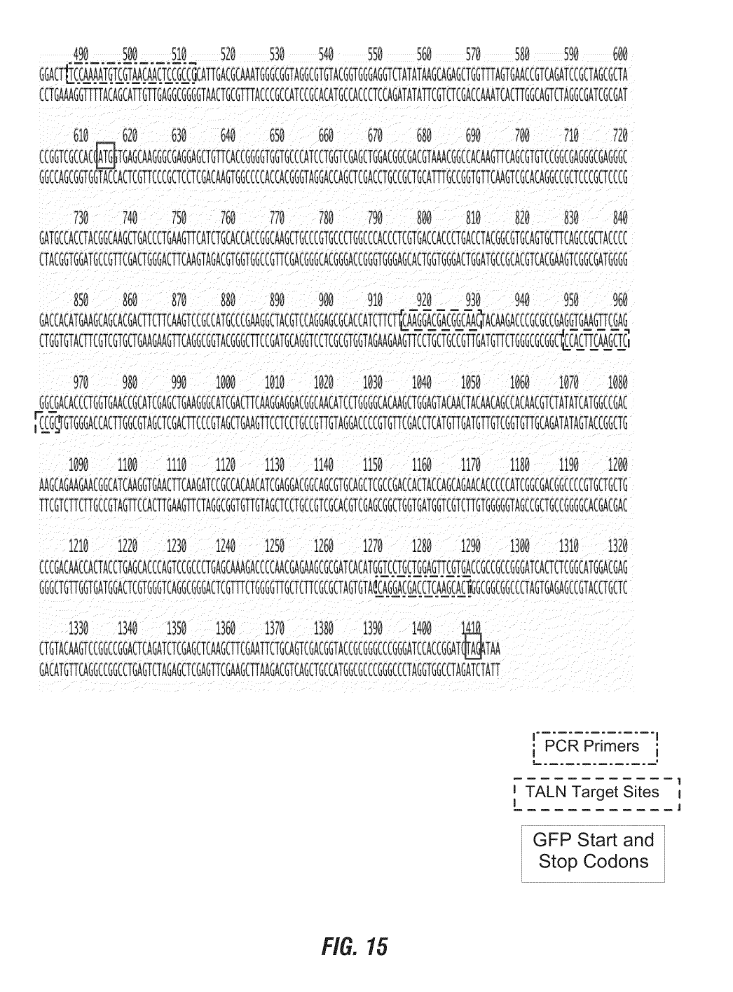

The GFP gene was amplified and sequenced from treated cells. Primers were designed for EGFP amplification, PCR reaction, and TA cloning of PCR product. The positive clones were screened and sequenced. FIG. 15 shows a representative sequence for design primers.

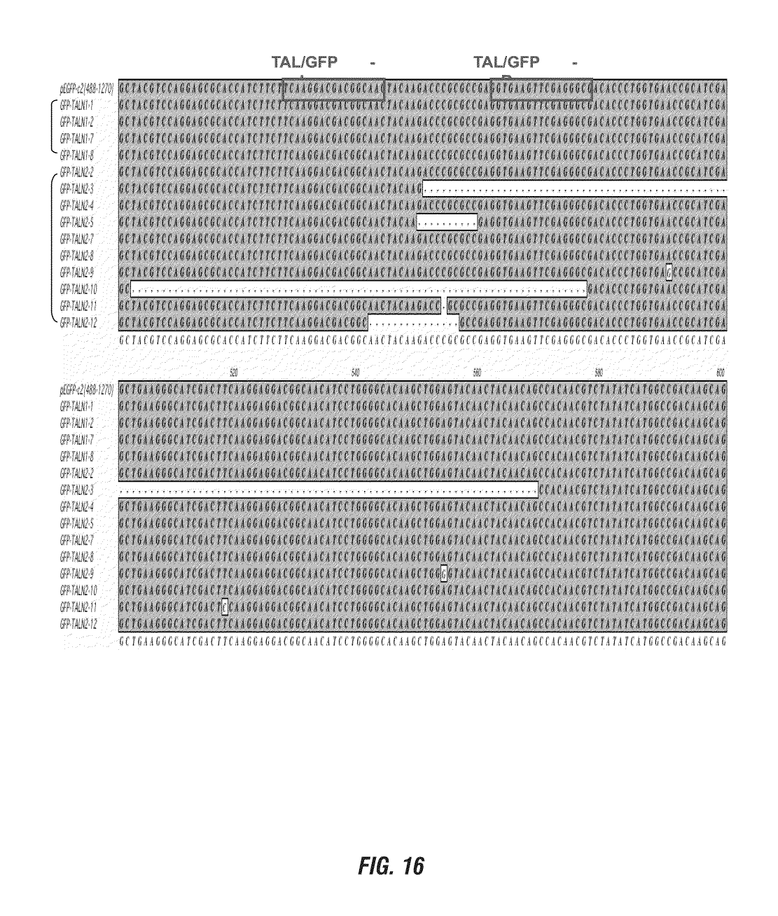

According to the results, targeted disruption of the GFP gene was observed. GFP-TAL1 (4 clones); 0 mg TALEN transfected; No insertions/deletions. GFP-TAL2 (10 clones); 0.5 mg/well TALEN (0.5 ug/well); 5/10 clones contain deletions at target site. The sequence results are depicted in FIG. 16.

The contents of any patents, patent applications, and references cited throughout this specification are hereby incorporated by reference in their entireties.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for targeted recombination in a cell at comprising:
introducing to said cell a fusion protein comprising a TAL type III effector binding domain and cleavage domain; so that cellular chromatin is cleaved in the region targeted by TAL effector binding domain so that homologous recombination may occur.

2. The method of claim 1 wherein said TAL type III effector binding domain has been modified to bind a site different from the non modified TAL type III effector binding domain.

3. The method of claim 1 wherein said TAL type III effector is from Xanthomonas oryzae pv. oryzae.

4. The method of claim 1 wherein said TAL type III effector is AvrXa7.

5. The method of claim 1 wherein said TAL type III effector activates the rice gene Os11N3.

6. The method of claim 1 wherein said cleavage domain is from FokI.

7. The method of claim 1 wherein the cellular chromatin is in a chromosome.

8. The method of claim 1 wherein, in the fusion protein, the cleavage domain is closer to the N-terminus and the TAL effector binding domain is closer to the C.

9. A fusion protein comprising a TAL type III effector sequence from Xanthomonas oryzae pv. oryzae and a FokI cleavage domain.

10. The fusion protein of claim 9 wherein said TAL type III effector sequence is AvrXa7.

11. The fusion protein if claim 10 wherein said AvrXa7 sequence has been modified to alter the target site.

12. The fusion protein of claim 10 wherein said fusion protein has the amino acid sequence of SEQ ID NO:1.

13. A nucleotide sequence encoding the amino acid sequence of claim 9.

14. A modified cell comprising the fusion protein of claim 9.

15. A vector comprising the nucleotide sequence of claim 13.

16. A method for targeted recombination in a cell at or near the rice gene Os11N3 comprising:
introducing to said cell a fusion protein comprising a TAL type II effector binding domain AvrXa7 and a cleavage domain FokI; so that cellular chromatin is cleaved in the region targeted by AvrXa7 so that homologous recombination may occur.

17. The method of claim 1 wherein said TAL type III effector is from Xanthomonas oryzae pv. oryzae.

18. The method of claim 1 wherein said TAL type III effector is AvrXa7.

19. The method of claim 1 wherein said TAL type III effector activates the rice gene Os11N3.

20. The method of claim 1 wherein said cleavage domain is from FokI.

21. The method of claim 1 wherein the cellular chromatin is in a chromosome.

22. The method of claim 1 wherein, in the fusion protein, the cleavage domain is closer to the N-terminus and the TAL effector binding domain is closer to the C.

23. A method for cleaving cellular chromatin in a region targeted by a TAL type III effector, the method comprising:
(a) selecting a region of interest;
(b) engineering a TAL type III.

24. A method for targeted recombination in a cell at comprising:
introducing to said cell a fusion protein comprising a AvrXa7 TAL type III effector binding domain target sequence and cleavage domain;
so that cellular chromatin is cleaved in the region targeted by TAL effector binding domain so that homologous recombination may occur.

25. The method of claim 24 wherein said AvrXa7 TAL type III effector binding domain targets the sequence ATAAACCCCCTCCAACCAGGTGCTAA.

26. The method of claim 24 wherein said AvrXa7 TAL type III effector is from Xanthomonas oryzae pv. oryzae.

27. The method of claim 24 wherein said binding domain target sequence is determined according to the following code of 12^{th} and 13^{th} amino acids of the AvrXa7 TAL type III effector binding domain:

| | |
|---|---|
| HD | C/G or A/T |
| NI | A/T or G/C or C/G |
| NG | T/A |
| NS | A/T or T/A or C/G |
| NN | C/G or A/T or G/C |
| N* | C/G or T/A or A/T |
| HG | T/A |

28. The method of claim 24 wherein said TAL type III effector activates the rice gene Os11N3.

29. The method of claim 24 wherein said cleavage domain is from FokI.

30. The method of claim 24 wherein the cellular chromatin is in a chromosome.

31. The method of claim 24 wherein, in the fusion protein, the cleavage domain is closer to the N-terminus and the TAL effector binding domain is closer to the C.

32. A fusion protein comprising:
a TAL type III effector sequence from Xanthomonas oryzae and a FokI cleavage domain.

33. The fusion protein of claim 32 wherein said TAL type III effector sequence is AvrXa7.

34. The fusion protein if claim 33 wherein said AvrXa7 sequence has been modified to alter the target site.

35. The fusion protein of claim 33 wherein said fusion protein has the amino acid sequence of SEQ ID NO:1.

36. A nucleotide sequence encoding the amino acid sequence of claim 32.

37. A modified cell comprising the fusion protein of claim 32.

38. A vector comprising the nucleotide sequence of claim 36.

39. A method for targeted recombination in a cell at or near the rice gene Os11N3 comprising:
introducing to said cell a fusion protein comprising a TAL type III effector binding domain AvrXa7 targeted sequence and a cleavage domain FokI;
so that cellular chromatin is cleaved in the region targeted by AvrXa7 so that homologous recombination may occur.

40. The method of claim 39 wherein said target sequence is determined according to the following code of 12^{th} and 13^{th} amino acids of the AvrXa7 target sequence:

| | |
|---|---|
| HD | C/G or A/T |
| NI | A/T or G/C or C/G |
| NG | T/A |
| NS | A/T or T/A or C/G |
| NN | C/G or A/T or G/C |
| N* | C/G or T/A or A/T |
| HG | T/A |

41. The method of claim 39 wherein said AvrXa7 TAL type III effector binding domain targets the sequence ATAAACCCCCTCCAACCAGGTGCTAA.

42. The method of claim 39 wherein said TAL type III effector activates the rice gene Os11N3.

43. The method of claim 39 wherein said cleavage domain is from FokI.

44. The method of claim 39 wherein the cellular chromatin is in a chromosome.

45. The method of claim 39 wherein, in the fusion protein, the cleavage domain is closer to the N-terminus and the TAL effector binding domain is closer to the C.

46. A method for cleaving cellular chromatin in a region targeted by a AvrXa7 TAL type III effector, the method comprising:
(a) selecting a region of interest;
(b) engineering a AvrXa7 TAL type III effector binding domain to bind to a first nucleotide sequence in the region of interest;
(c) expressing a first fusion protein in the cell, the first fusion protein comprising the AvrXa7 TAL type III effector binding domain and a cleavage domain;
wherein (i) the fusion protein binds to the first nucleotide sequence, such that the cellular chromatin is cleaved in the region of interest so that homologous recombination may occur.


SEQUENCE LISTING (94 sequences)

SEQ ID NO: 1 - protein, 33 aa, Xanthomonas oryzae
LTPDQVVAIASNIGGKQALETVQRLLPVLCQDH

SEQ ID NO: 2 - protein, 52 aa, Xanthomonas oryzae
  misc_feature (24)..(24): Xaa can be any naturally occurring amino acid
NIHGNININSHDNNHDHDHDNSNXNXHDHDNSNSNNNNNINGNNNINXNSNX

SEQ ID NO: 3 - DNA, 35 bp, Oryza sativa
gcactatataaaccccctccaaccaggtgctaagc

SEQ ID NO: 4 - DNA, 35 bp, Oryza sativa
gcacttttttttccccctccaaccaggtgctaagc

SEQ ID NO: 5 - DNA, 35 bp, oryza sativa
actatataaaccccctccaaccaggtgctaagctc

SEQ ID NO: 6 - DNA, 41 bp, Oryza sativa
actatcccgggataaaccccctccaaccaggtgctaagctc

SEQ ID NO: 7 - DNA, 60 bp, Oryza sativa
tataaaccccctccaaccaggtgctaagctcatcaagccttcaagcaaagcaaactcaag

SEQ ID NO: 8 - DNA, 53 bp, Oryza sativa
cattcccttcttccttcctagcactatataaaccccctccaaccaggtgctaa

SEQ ID NO: 9 - DNA, 4987 bp, Xanthomonas oryzae
agatctcagctagtgaaatctgaattggaagagaagaaatctgaacttagacataaattgaaatatgtgccacatgaatatattgaattgattgaaatcgcaagaaattcaactcaggatagaatccttgaaatgaaggtgatggagttctttatgaaggtttatggttatcgtggtaaacatttgggtggatcaaggaaaccagacggagcaatttatactgtcggatctcctattgattacggtgtgatcgttgatactaaggcatattcaggaggttataatcttccaattggtcaagcagatgaaatgcaaagatatgtcgaagagaatcaaacaagaaacaagcatatcaaccctaatgaatggtggaaagtctatccatcttcagtaacagaatttaagttcttgtttgtgagtggtcatttcaaaggaaactacaaagctcagcttacaagattgaatcatatcactaattgtaatggagctgttcttagtgtagaagagcttttgattggtggagaaatgattaaagctggtacattgacacttgaggaagtgagaaggaaatttaataacggtgagataaactttggatctgccaccatggatcccattcgttcgcgcacgccaagtcctgcccgcgagcttctgcccggaccccaaccggatagggttcagccgactgcagatcgggggggggctccgcctgctggcggccccctggatggcttgcccgctcggcggacgatgtcccggacccggctgccatctccccctgcgccctcgcctgcgttctcggcgggcagcttcagcgatctgctccgtcagttcgatccgtcgcttcttgatacatcgcttcttgattcgatgcctgccgtcggcacgccgcatacagcggctgccccagcagagtgggatgaggtgcaatcgggtctgcgtgcagccgatgacccgccacccaccgtgcgtgtcgctgtcactgccgcgcggccgccgcgcgccaagccggccccgcgacggcgtgcggcgcaaccctccgacgcttcgccggccgcgcaggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcaagtatcagcacataatcacggcgttgccagaggcgacacacgaagacatcgttggcgtcggcaaacagtggtccggcgcacgcgccctggaggccttgctcacgaaggcgggggagttgagaggtccgccgttacagttggacacaggccaacttctcaagattgcaaaacgtggcggcgtgaccgcagtggaggcagtgcatgcatggcgcaatgcactgacgggtgcccccctgaacctgaccccggaccaagtggtggccatcgccagcaatattggcggcaagcaggcgctggagacggtacagcggctgttgccggtgctgtgccaggaccatggcctgaccccggaccaggtcgtggccatcgccagccatggcggcggcaagcaggcgctggagacggtgcagcggctgttgccggtgctgtgccaggaccatggcctgaccccggaccaggtggtggccatcgccagcaatattggcggcaagcaggcgctagagacggtgcagcggctgttgccggtgctgtgccaggcccatggcctgaccccggaccaggtcgtggccatcgccagcaatattggcggcaagcaggcgctggagacggtgcagcggctgttgccggtgctgtgccaggaccatggcctgaccccggcccaggtggtggccatcgccagcaatagtggcggcaagcaggcgctggagacggtgcagcggctgttgccggtgctgtgccaggaccatggcctgaccccggaccaagtcgtggccatcgccagccacgatggcggcaagcaggcgctggagacgctgcagcggctgttgccggtgctgtgccaggaccatggcctgaccccggaccaggtcgtggccatcgccaacaataacggcggcaagcaggcgctggagacgctgcagcggctgttgccggtgctgtgccaggaccatggcctgaccccggaccaagtggtggccatcgccagccacgatggcggcaagcaggcgctggagacggtgcagcggctgttgccggtgctgtgccaggaccatggcctgaccccggaccaggtggtggccatcgccagccacgatggcggcaagcaggcgctggagacggtgcagcggctgttgccggtgctgtgccaggaccatggcctgaccccggcccaagtggtggccatcgccagccacgatggcggcaagcaggcgctggagacggtgcagcggctgttgccggtgctgtgccaggaccatggcctgaccccggaccaggtggtggccatcgccagcaatagcggcggcaagcaggcgctggagacggtacagcggctgttgccggtgctgtgccaggaccatggactgaccccggaccaggtcgtggccatcgccagcaatggcggcaagcaggcgctggagacggtacagcggctgttgccggtgctgtgccaggaccatggcctgaccccggaccaggtcgtggccatcgccagcaatggcggcaagcaggcgctggagacggtgcagcggctgttgccggtacagcggctgttgccggtgctgtgccaggaccatggcctgacccaggaccaggtggtggccatcgccagccacgatggcggcaagcaggcgctggagacggtgcagcggctgttgccggtgctgtgccaggaccatggcctgaccccggaccaagtggtggccatcgccagccacgatggcggcaaacaggcgctggagacggtgcagcggctgttgccggtgctgtgccaggaccatggcctgaccccggaccaggtggtggccatcgccagcaatagtggcggcaagcaggcgctggagacggtgcagcggctgttgccggtgctgtgccaggaccatggcctgaccccggaccaagtggtggccatcgccagcaatagtggcggcaagcaggcgctggagacggtgcagcggctgttgccggtgctgtgccaggaccatggcctgaccccggaccaggtggtggccatcgccagcaataacggcggcaagcaggcgctggagacggtgcagcggctgttgccggtgctgtgccaggaccatggcctgaccccggaccaggtcgtggccatcgccaacaataacggcggcaagcaggcgctggagacggtgcagcggctgttgccggtgctgtgccaggaccatggcctgaccccggcgcaggtggtggccatcgccagcaatattggcggcaagcaggcgctggagacggtgcagcggctgttgccggtgctgtgccaggaccatggcctgaccctggaccaggtggtggccattgccagcaatggcggcagcaaacaggcgctagagacggtgcagcggctgttgccggtgctgtgccaggaccatggcctgaccccggaccaagtggtggccatcgccaacaataacggcggcaagcaggcgctggagacggtgcagcggctgttgccggtgctgtgccaggaccatggcctgaccccggaccaggtcgtggccatcgccagcaatattggcggcaagcaggcgctggagacggtgcagcggctgttgccggtgctgtgccaggaccatggcctgaccctggaccaggtggtggccatcgccagcaatggcggcaagcaggcgctggagacggtgcagcggctgttgccggtgctgtgccaggaccatggcctgaccccgaaccaggtggtggccatcgccagcaatagtggcggcaagcaggcgctggagacggtgcagcggctgttgccggtgctgtgccaggaccatggcctgaccccgaaccaggtggtggccatcgccagcaatggcggcaagcaggcgctggagagcattgttgcccagttatctcgccctgatccggcgttggccgcgttgaccaacgaccacctcgtcgccttggcctgcctcggcggacgtcctgccctggatgcagtgaaaaagggattgccgcacgcgccggaattgatcagaagaatcaatcgccgcattcccgaacgcacgtcccatcgcgttcccgacctcgcgcacgtggttcgcgtgcttggttttttccagagccactcccacccagcgcaagcattcgatgacgccatgacgcagttcgagatgagcaggcacggcttggtacagctctttcgcagagtgggcgtcaccgaattcgaagcccgctacggaacgctccccccagcctcgcagcgttgggaccgtatcctccaggcatcagggatgaaaagggccaaaccgtcccctacttcagctcaaacaccggatcaggcgtctttgcatgcagattacaaggacgacgacgacaagaaggattacaaggacgacgacgacaagaagggtcgacccagcccaatgcacgagggagatcagacgcgggcaagcagccgtaaacggtcccgatcggatcgtgctgtcaccggcccctccacacagcaatctttcgaggtgcgcgttcccgaacagcaagatgcgctgcatttgcccctcagctggagggtaaaacgcccgcgtaccaggatcgggggcggcctcccggatcctggtacgcccatcgctgccgacctggcagcgtccagcaccgtgatgtgggaacaagatgcggcccccttcgcaggggcagcggatgatttcccggcattcaacgaagaggagctcgcatggttgatggagctattgcctcagtcaggctcagtcggagggacgatcaagcttctagactagt

SEQ ID NO: 10 - protein, 1661 aa, Xanthomonas oryzae
RSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSATMDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAEWDEVQSGLRAADDPPPTVRVAVTAARPPRAKPAPRRRAAQPSDASPAAQVDLRTLGYSQQQQEKIKPKVRSTVAQHHEALVGHGFTHAHIVALSQHPAALGTVAVKYQHIITALPEATHEDIVGVGKQWSGARALEALLTKAGELRGPPLQLDTGQLLKIAKRGGVTAVEAVHAWRNALTGAPLNLTPDQVVAIASNIGGKQALETVQRLLPVLCQDHGLTPDQVVAIASHGGGKQALETVQRLLPVLCQDHGLTPDQVVAIASNIGGKQALETVQRLLPVLCQAHGLTPDQVVAIASNIGGKQALETVQRLLPVLCQDHGLTPAQVVAIASNSGGKQALETVQRLLPVLCQDHGLTPDQVVAIASHDGGKQALETLQRLLPVLCQDHGLTPDQVVAIANNNGGKQALETLQRLLPVLCQDHGLTPDQVVAIASHDGGKQALETVQRLLPVLCQDHGLTPDQVVAIASHDGGKQALETVQRLLPVLCQDHGLTPAQVVAIASHDGGKQALETVQRLLPVLCQDHGLTPDQVVAIASNSGGKQALETVQRLLPVLCQDHGLTPDQVVAIASNGGKQALETVQRLLPVLCQDHGLTPDQVVAIASNGGKQALETVQRLLPVQRLLPVLCQDHGLTQDQVVAIASHDGGKQALETVQRLLPVLCQDHGLTPDQVVAIASHDGGKQALETVQRLLPVLCQDHGLTPDQVVAIASNSGGKQALETVQRLLPVLCQDHGLTPDQVVAIASNSGGKQALETVQRLLPVLCQDHGLTPDQVVAIASNNGGKQALETVQRLLPVLCQDHGLTPDQVVAIANNNGGKQALETVQRLLPVLCQDHGLTPAQVVAIASNIGGKQALETVQRLLPVLCQDHGLTLDQVVAIASNGGSKQALETVQRLLPVLCQDHGLTPDQVVAIANNNGGKQALETVQRLLPVLCQDHGLTPDQVVAIASNIGGKQALETVQRLLPVLCQDHGLTLDQVVAIASNGGKQALETVQRLLPVLCQDHGLTPNQVVAIASNSGGKQALETVQRLLPVLCQDHGLTPNQVVAIASNGGKQALESIVAQLSRPDPALAALTNDHLVALACLGGRPALDAVKKGLPHAPELIRRINRRIPERTSHRVPDLAHVVRVLGFFQSHSHPAQAFDDAMTQFEMSRHGLVQLFRRVGVTEFEARYGTLPPASQRWDRILQASGMKRAKPSPTSAQTPDQASLHADYKDDDDKKDYKDDDDKKGRPSPMHEGDQTRASSRKRSRSDRAVTGPSTQQSFEVRVPEQQDALHLPLSWRVKRPRTRIGGGLPDPGTPIAADLAASSTVMWEQDAAPFAGAADDFPAFNEEELAWLMELLPQSGSVGGTIKLLD

SEQ ID NO: 11 - DNA, 39 bp, Xanthomonas oryzae
agatctatataaaccccctccaaccaggtgctaactagt

SEQ ID NO: 12 - DNA, 83 bp, Xanthomonas oryzae
agatcttagcacctggttggagggggtttatatagtgctaggaaggtaccctataaaccccctccaaccaggtgctaactagt

SEQ ID NO: 13 - DNA, 88 bp, Xanthomonas oryzae
agatcttagcacctggttggagggggtttatatagtgctaggaaggttcggtaccctataaaccccctccaaccaggtgctaactagt

SEQ ID NO: 14 - DNA, 94 bp, Xanthomonas oryzae
agatcttagcacctggttggagggggtttatatagtgctaggaaggaagaacttcggtaccctataaaccccctccaaccaggtgctaactagt

SEQ ID NO: 15 - DNA, 804 bp, Saccharomyces cerevisiae
atgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 16 - DNA, 828 bp, Saccharomyces cerevisiae
atgcgcccacgcggatccgcagaagcctcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 17 - DNA, 874 bp, Saccharomyces cerevisiae
atgcatctccccctactgtacaccaccaaaagtgaattcatgagctttagcacctggttggagggggtttatatcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 18 - DNA, 58 bp, Saccharomyces cerevisiae
taaatcatgcgcccacgcggatcgatccgcagaagcctcgaaagctacatataaggaa

SEQ ID NO: 19 - DNA, 54 bp, Saccharomyces cerevisiae
taaatcatgcgcccacgcggatccgcagaagcctcgaaagctacatataaggaa

SEQ ID NO: 20 - DNA, 46 bp, Saccharomyces cerevisiae
taaatcatgcgcccacgcagaagcctcgaaagctacatataaggaa

SEQ ID NO: 21 - DNA, 46 bp, Saccharomyces cerevisiae
taaatcatgcgcccacgcggaagcctcgaaagctacatataaggaa

SEQ ID NO: 22 - DNA, 34 bp, Saccharomyces cerevisiae
taaatcatgcgcctcgaaagctacatataaggaa

SEQ ID NO: 23 - DNA, 37 bp, Saccharomyces cerevisiae
taaatcatgcgcccacgcgaaagctacatataaggaa

SEQ ID NO: 24 - DNA, 85 bp, Saccharomyces cerevisiae
aaatcatgcatctccccctactgtacaccaccaaaagtgaattcatgtgagcttagcacctggttggagggggtttatatcgaaa

SEQ ID NO: 25 - DNA, 85 bp, Saccharomyces cerevisiae
aaatcatgcatctccccctactgtacaccaccaaaagtgaattcatgagcttttagcacctggttggagggggtttatatcgaaa

SEQ ID NO: 26 - DNA, 85 bp, Saccharomyces cerevisiae
aaatcatgcatctccccctactgtacaccaccaaaagtgaattcatgagagcttagcacctggttggagggggtttatatcgaaa

SEQ ID NO: 27 - DNA, 84 bp, Saccharomyces cerevisiae
aaatcatgcatctccccctactgtacaccaccaaaagtgaattcatgagctttagcacctggttggagggggtttatatcgaaa

SEQ ID NO: 28 - DNA, 83 bp, Saccharomyces cerevisiae
aaatcatgcatctccccctactgtacaccaccaaaagtgaattcatgagcttagcacctggttggagggggtttatatcgaaa

SEQ ID NO: 29 - DNA, 57 bp, Saccharomyces cerevisiae
aaatcatgcatctccccctactgtacaccacctggttggagggggtttatatcgaaa

SEQ ID NO: 30 - DNA, 54 bp, Saccharomyces cerevisiae
aaatcatgcatctccccctactgtacacctggttggagggggtttatatcgaaa

SEQ ID NO: 31 - DNA, 102 bp, Xanthomonas oryzae
cgctggagacggtacagcggctgttgccggtgctgtgccaggaccatggcctgaccccggaccaagtggtggccatcgccagcaatattggcggcaagcagg

SEQ ID NO: 32 - DNA, 102 bp, Xanthomonas oryzae
cgctggagacggtacagcggctgttgccggtgctgtgccaggaccatggcctgaccccggaccaggtcgtggccatcgccagccatggcggcggcaagcagg

SEQ ID NO: 33 - DNA, 102 bp, Xanthomonas oryzae
cgctggagacggtacagcggctgttgccggtgctgtgccaggaccatggcctgaccccggaccaggtcgtggccatcgccaacaataacggcggcaagcagg

SEQ ID NO: 34 - DNA, 102 bp, Xanthomonas oryzae
cgctggagacggtacagcggctgttgccggtgctgtgccaggaccatggcctgaccccggaccaagtcgtggccatcgccagccacgatggcggcaagcagg

SEQ ID NO: 35 - DNA, 804 bp, Saccharomyces cerevisiae; identical to SEQ ID NO: 15

SEQ ID NO: 36 - protein, 32 aa, Xanthomonas oryzae
NINGNININNNNNINIHDNNNGNNHDNGNNHD

SEQ ID NO: 37 - DNA, 16 bp, Xanthomonas oryzae
ataaggaacgtgctgc

SEQ ID NO: 38 - protein, 36 aa, Xanthomonas oryzae
NINGNGNINININGNINNHDNGNGNNNNHDNINGHD

SEQ ID NO: 39 - DNA, 18 bp, Xanthomonas oryzae
attaaatagcttggcagc

SEQ ID NO: 40 - protein, 32 aa, Xanthomonas oryzae
NINGNNNIHDNIHDHDHDNNNNNGNNNNNNNN

SEQ ID NO: 41 - DNA, 16 bp, Xanthomonas oryzae
atgacacccggtgtgg

SEQ ID NO: 42 - protein, 32 aa, Xanthomonas oryzae
NIHDNGNNNGNGNNNINNHDHDNININGNNHD

SEQ ID NO: 43 - DNA, 16 bp, Xanthomonas oryzae
actgttgacccaatgc

SEQ ID NO: 44 - DNA, 64 bp, Saccharomyces cerevisiae
tacatataaggaacgtgctgctactcatcctatagtcctgttgctgccaagctatttaatatca

SEQ ID NO: 45 - DNA, 67 bp, Saccharomyces cerevisiae
tacatataaggaacgtgctgctactcatccctagtcctgttgctgccaagctatttaatatcatgca

SEQ ID NO: 46 - DNA, 66 bp, Saccharomyces cerevisiae
tacatataaggaacgtgctgctactcatcctagtcctgttgctgccaagctatttaatatcatgca

SEQ ID NO: 47 - DNA, 65 bp, Saccharomyces cerevisiae
tacatataaggaacgtgctgctactcatctagtcctgttgctgccaagctatttaatatcatgca

SEQ ID NO: 48 - DNA, 54 bp, Saccharomyces cerevisiae
tacatataaggaacgtgctagtcctgttgctgccaagctatttaatatcatgca

SEQ ID NO: 49 - DNA, 42 bp, Saccharomyces cerevisiae
tacatataaggaacgtgctgccaagctatttaatatcatgca

SEQ ID NO: 50 - DNA, 63 bp, Saccharomyces cerevisiae
tgattatgacacccggtgtggggtttagatgacaagggagacgcattgggtcaacagtataga

SEQ ID NO: 51 - DNA, 62 bp, Saccharomyces cerevisiae
tgattatgacacccggtgtgggtttagatgacaagggagacgcattgggtcaacagtataga

SEQ ID NO: 52 - DNA, 47 bp, Saccharomyces cerevisiae
tgattatgacacccggtgtgggagacgcattgggtcaacagtataga

SEQ ID NO: 53 - DNA, 54 bp, Saccharomyces cerevisiae
tgattatgacacccggtgtgggtttagggagacgcattgggtcaacagtataga

SEQ ID NO: 54 - DNA, 53 bp, Xanthomonas oryzae
ttcaaggacgacggcaactacaagacccgcgccgaggtgaagttcgagggcga

SEQ ID NO: 55 - DNA, 17 bp, Xanthomonas oryzae
tcaaggacgacggcaac

SEQ ID NO: 56 - protein, 34 aa, Xanthomonas oryzae
  misc_feature (34)..(34): Xaa can be any naturally occurring amino acid
HGHDNININNNNNIHDNNNIHDNNNNHDNININX

SEQ ID NO: 57 - DNA, 16 bp, Xanthomonas oryzae
cgccctcgaacttcac

SEQ ID NO: 58 - protein, 32 aa, Xanthomonas oryzae
HDNNHDHDHDHGHDNNNINIHDHGHGHDNIHD

SEQ ID NO: 59 - DNA, 856 bp, Xanthomonas oryzae
ggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaataa

SEQ ID NO: 60 - DNA, 200 bp, Xanthomonas oryzae
gctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcag

SEQ ID NO: 61 - DNA, 200 bp, Xanthomonas oryzae; identical to SEQ ID NO: 60

SEQ ID NO: 62 - DNA, 200 bp, Xanthomonas oryzae; identical to SEQ ID NO: 60

SEQ ID NO: 63 - DNA, 200 bp, Xanthomonas oryzae; identical to SEQ ID NO: 60

SEQ ID NO: 64 - DNA, 81 bp, Xanthomonas oryzae
gctacgtccaggagcgcaccatcttcttcaaggacgacggcaactacaagccacaacgtctatatcatggccgacaagcag

SEQ ID NO: 65 - DNA, 200 bp, Xanthomonas oryzae; identical to SEQ ID NO: 60

SEQ ID NO: 66 - DNA, 190 bp, Xanthomonas oryzae
gctacgtccaggagcgcaccatcttcttcaaggacgacggcaactacaagaggtgaagttcgagggcgacaccctggtgaaccgcatcgagctgaagggcatcgacttcaaggaggacggcaacatcctggggcacaagctggagtacaactacaacagccacaacgtctatatcatggccgacaagcag

SEQ ID NO: 67 - DNA, 200 bp, Xanthomonas oryzae; identical to SEQ ID NO: 60

SEQ ID NO: 68 - DNA, 200 bp, Xanthomonas oryzae; identical to SEQ ID NO: 60

SEQ ID NO: 69 - DNA, 200 bp, Xanthomonas oryzae
gctacgtccaggagcgcaccatcttcttcaaggacgacggcaactacaagacccgcgccgaggtgaagttcgagggcgacaccctggtgagccgcatcgagctgaagggcatcgacttcaaggaggacggcaacatcctggggcacaagctggggtacaactacaacagccacaacgtctatatcatggccgacaagcag

SEQ ID NO: 70 - DNA, 125 bp, Xanthomonas oryzae
gcgacaccctggtgaaccgcatcgagctgaagggcatcgacttcaaggaggacggcaacatcctggggcacaagctggagtacaactacaacagccacaacgtctatatcatggccgacaagcag

SEQ ID NO: 71 - DNA, 199 bp, Xanthomonas oryzae
gctacgtccaggagcgcaccatcttcttcaaggacgacggcaactacaagaccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcag

SEQ ID NO: 72 - DNA, 185 bp, Xanthomonas oryzae
gctacgtccaggagcgcaccatcttcttcaaggacgacggcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcag

SEQ ID NO: 73 - DNA, 38 bp, Xanthomonas oryzae
ccatggtaccagatctcagctagtgaaatctgaattgg

SEQ ID NO: 74 - DNA, 36 bp, Xanthomonas oryzae
ccggatccaaagtttatctcaccgttattaaatttc

SEQ ID NO: 75 - DNA, 31 bp, Xanthomonas oryzae
caccagatctcgccaccatggtgagcaaggg

SEQ ID NO: 76 - DNA, 29 bp, Xanthomonas oryzae
ccggatcctccggacttgtacagctcgtc

SEQ ID NO: 77 - DNA, 45 bp, Xanthomonas oryzae
caccggtaccagatctgccaccatggatcccattcgttcgcgcac

SEQ ID NO: 78 - DNA, 41 bp, Xanthomonas oryzae
ccactagtctagaagcttgatcgtccctccgactgagcctg

SEQ ID NO: 79 - DNA, 35 bp, Xanthomonas oryzae; identical to SEQ ID NO: 3

SEQ ID NO: 80 - DNA, 35 bp, Xanthomonas oryzae
gcttagcacctggttggagggggtttatatagtgc

SEQ ID NO: 81 - DNA, 35 bp, Xanthomonas oryzae; identical to SEQ ID NO: 4

SEQ ID NO: 82 - DNA, 35 bp, Xanthomonas oryzae
gcttagcacctggttggagggggaaaaaaaagtgc

SEQ ID NO: 83 - DNA, 29 bp, Xanthomonas oryzae
caccggtaccatggctgtgattgatcagg

SEQ ID NO: 84 - DNA, 29 bp, Xanthomonas oryzae
ccggatccagccattgcagcaagatcttg

SEQ ID NO: 85 - DNA, 33 bp, Xanthomonas oryzae
gatctatataaaccccctccaaccaggtgctaa

SEQ ID NO: 86 - DNA, 33 bp, Xanthomonas oryzae
ctagttagcacctggttggagggggtttatata

SEQ ID NO: 87 - DNA, 48 bp, Xanthomonas oryzae
gatcttagcacctggttggagggggtttatatagtgctaggaaggtac

SEQ ID NO: 88 - DNA, 40 bp, Xanthomonas oryzae
cttcctagcactatataaaccccctccaaccaggtgctaa

SEQ ID NO: 89 - DNA, 53 bp, Xanthomonas oryzae
gatcttagcacctggttggagggggtttatatagtgctaggaaggttcggtac

SEQ ID NO: 90 - DNA, 45 bp, Xanthomonas oryzae
cgaaccttcctagcactatataaaccccctccaaccaggtgctaa

SEQ ID NO: 91 - DNA, 58 bp, Xanthomonas oryzae
gatctgtggtgtacagtagggggagatgcatatctaacctttgcttttttttcggtac

SEQ ID NO: 92 - DNA, 49 bp, Xanthomonas oryzae
cgaaaaaaaagcaaaggttagatatgcatctccccctactgtacaccac

SEQ ID NO: 93 - DNA, 30 bp, Xanthomonas oryzae
ctagaggatccgtcgacaagcttactagtc

SEQ ID NO: 94 - DNA, 30 bp, Xanthomonas oryzae
tcgagactagtaagcttgtcgacggatcct